(19) United States
(12) Patent Application Publication
(10) Pub. No.: US 2024/0002944 A1
(43) Pub. Date: Jan. 4, 2024

(54) METHODS AND GENOMIC CLASSIFIERS FOR IDENTIFYING HOMOLOGOUS RECOMBINATION DEFICIENCY PROSTATE CANCER

(71) Applicants: DECIPHER BIOSCIENCES, INC., San Diego, CA (US); Northwestern University, Evanston, IL (US)

(72) Inventors: Edward Matthew Schaeffer, Winnetka, IL (US); Elai Davicioni, San Diego, CA (US); Yang Liu, Vancouver (CA); Adam Benjamin Weiner, Chicago, IL (US)

(21) Appl. No.: 18/037,885

(22) PCT Filed: Nov. 18, 2021
(86) PCT No.: PCT/US2021/059873

§ 371 Date: May 19, 2023

Related U.S. Application Data
(60) Provisional application No. 63/116,734, filed on Nov. 20, 2020.

(51) Int. Cl. C12Q 1/6886; G16B 25/10; G16H 20/10; G16H 50/20

(52) U.S. Cl. CPC C12Q 1/6886; G16B 25/10; G16H 20/10; G16H 50/20; C12Q 2600/106; C12Q 2600/112; C12Q 2600/158; C12Q 2600/16

(57) ABSTRACT

The disclosure relates to methods, systems, kits and probe sets for the identification, determination, diagnosis, and/or prognosis of homologous recombination deficiency prostate cancer in a subject. The disclosure also provides biomarkers and clinically useful genomic classifiers for identifying homologous recombination deficiency prostate cancer, bioinformatic methods for determining clinically useful classifiers, and methods of use of each of the foregoing. The methods, systems, kits and probe sets can provide expression-based analysis of biomarkers for purposes of homologous recombination deficiency prostate cancer in a subject. Methods of treating homologous recombination deficiency prostate cancer based on expression analysis are also provided. The methods and classifiers of the present disclosure are also useful for predicting response to anticancer therapy (e.g., PARP inhibitors).

### CROSS-REFERENCE TO RELATED APPLICATIONS

This application claims priority to U.S. Ser. No. 63/116,734, filed Nov. 20, 2020, which is herein incorporated by reference in its entirety.

### FIELD OF THE INVENTION

The disclosure relates to methods, systems, kits and probe sets for the identification, determination, diagnosis, and/or prognosis of homologous recombination deficiency prostate cancer in a subject. The disclosure also provides biomarkers and clinically useful genomic classifiers for identifying homologous recombination deficiency prostate cancer, bioinformatic methods for determining clinically useful classifiers, and methods of use of each of the foregoing. The methods, systems, kits and probe sets can provide expression-based analysis of biomarkers for purposes of homologous recombination deficiency prostate cancer in a subject. Methods of treating homologous recombination deficiency prostate cancer based on expression analysis are also provided. The methods and classifiers of the present disclosure are also useful for predicting response to anticancer therapy (e.g., PARP inhibitors).

### BACKGROUND OF THE INVENTION

Cancer is the uncontrolled growth of abnormal cells anywhere in a body. The abnormal cells are termed cancer cells, malignant cells, or tumor cells. Many cancers and the abnormal cells that compose the cancer tissue are further identified by the name of the tissue that the abnormal cells originated from (for example, prostate cancer). Cancer cells can proliferate uncontrollably and form a mass of cancer cells. Cancer cells can break away from this original mass of cells, travel through the blood and lymph systems, and lodge in other organs where they can again repeat the uncontrolled growth cycle. This process of cancer cells leaving an area and growing in another body area is often termed metastatic spread or metastatic disease. For example, if prostate cancer cells spread to a bone (or anywhere else), it can mean that the individual has metastatic prostate cancer.

Standard clinical parameters such as tumor size, grade, lymph node involvement and tumor-node-metastasis (TNM) staging (American Joint Committee on Cancer http://www.cancerstaging.org) may correlate with outcome and serve to stratify patients with respect to (neo)adjuvant chemotherapy, immunotherapy, antibody therapy and/or radiotherapy regimens. Incorporation of molecular markers in clinical practice may define tumor subtypes that are more likely to respond to targeted therapy. However, stage-matched tumors grouped by histological or molecular subtypes may respond differently to the same treatment regimen.

Additional key genetic and epigenetic alterations may exist with important etiological contributions. A more detailed understanding of the molecular mechanisms and regulatory pathways at work in cancer cells and the tumor microenvironment (TME) could dramatically improve the design of novel anti-tumor drugs and inform the selection of optimal therapeutic strategies. The development and implementation of diagnostic, prognostic and therapeutic biomarkers to characterize the biology of each tumor may assist clinicians in making important decisions with regard to individual patient care and treatment.

This background information is provided for the purpose of making known information believed by the applicant to be of possible relevance to the present disclosure. No admission is necessarily intended, nor should be construed, that any of the preceding information constitutes prior art against the present disclosure.

### SUMMARY OF THE INVENTION

Thus, provided herein are methods, systems and kits for the diagnosis, prognosis and the determination of cancer progression of cancer in a subject. The present disclosure relates to methods, systems and kits for the diagnosis, prognosis and the determination of homologous recombination deficiency prostate cancer in a subject. The disclosure also provides biomarkers that identify homologous recombination deficiency prostate cancer, clinically useful classifiers for identifying homologous recombination deficiency prostate cancer, bioinformatic methods for determining clinically useful classifiers, and methods of use of each of the foregoing. The methods, systems and kits can provide expression-based analysis of biomarkers for purposes of identifying homologous recombination deficiency prostate cancer in a subject. Further disclosed herein, in certain instances, are probe sets for use in identifying homologous recombination deficiency prostate cancer in a subject. Classifiers for identifying homologous recombination deficiency prostate cancer are provided. Methods of treating homologous recombination deficiency prostate cancer based on expression analysis are also provided.

An aspect of the present disclosure is a method comprising: obtaining a biological sample from a subject having prostate cancer, wherein the sample comprises nucleic acids; and detecting the level of expression of a plurality of targets selected from Table 6 or Table 7. An aspect of the present disclosure is a method comprising: a) obtaining or having obtained a nucleic acid expression level of a plurality of targets selected from Table 6 or Table 7, in a biological sample from a subject having prostate cancer; b) prognosing the patient with homologous recombination deficiency prostate cancer based on the nucleic acid expression levels; and c) administering an effective amount of a treatment to the patient based on the prognosis, wherein the treatment is a PARP inhibitor. An aspect of the present disclosure is a method comprising: a) obtaining or having obtained a nucleic acid expression level of a plurality of targets selected from Table 6 or Table 7, in a biological sample from a subject having prostate cancer; b) determining that the patient has homologous recombination deficiency prostate cancer based on the nucleic acid expression levels; and c) administering an effective amount of a treatment to the subject determined to have homologous recombination deficiency prostate cancer; based on the nucleic acid expression levels, wherein the treatment is a PARP inhibitor. In an embodiment, the method further comprises administering an anti-cancer treatment other than a PARP inhibitor to the subject if the expression levels indicate that the subject does not have homologous recombination deficiency prostate cancer. In an embodiment, the anti-cancer treatment other than a PARP inhibitor is selected from the group consisting of surgery, chemotherapy, radiation therapy, immunotherapy, biological therapy, neoadjuvant chemotherapy, and photodynamic therapy. In an embodiment, the expression level of said target is reduced expression of said target. In an embodiment, the expression level of said target is increased expression of said target. In an embodiment, the level of expression of said target is determined by using a method selected from the group consisting of in situ hybridization, a PCR-based method, an array-based method, an immunohistochemical method, an RNA assay method and an immunoassay method. In an embodiment, the method further comprises determining the level of expression of said plurality of targets using at least one reagent that specifically binds to said targets. In an embodiment, the reagent is selected from the group consisting of a nucleic acid probe, one or more nucleic acid primers, and an antibody. In an embodiment, the target comprises a nucleic acid sequence. In an embodiment, the biological sample is a biopsy. In an embodiment, the biological sample is a urine sample, a blood sample or a prostate tumor sample. In an embodiment, the blood sample is plasma, serum, or whole blood. In an embodiment, the subject is a human. In an embodiment, the measuring the level of expression comprises measuring the level of an RNA transcript. In an embodiment, the method further comprises administering at least one cancer treatment selected from the group consisting of surgery, radiation therapy, immunotherapy, biological therapy, neoadjuvant chemotherapy, and photodynamic therapy after the androgen deprivation therapy.

An aspect of the disclosure is a kit for identifying, diagnosing and/or prognosing prostate cancer in a subject, the kit comprising agents for detecting the presence or expression levels for a plurality of targets, wherein said plurality of genes comprises one or more genes selected from Table 6 or Table 7. In an embodiment, the agents comprise reagents for performing in situ hybridization, a PCR-based method, an array-based method, a sequencing method, an immunohistochemical method, an RNA assay method, or an immunoassay method. In an embodiment, the agents comprise one or more of a microarray, a nucleic acid probe, a nucleic acid primer, or an antibody. In an embodiment, the kit comprises at least one set of PCR primers capable of amplifying a nucleic acid comprising a sequence of a gene selected from Table 6 or Table 7 or its complement. In an embodiment, the kit comprises at least one probe capable of hybridizing to a nucleic acid comprising a sequence of a gene selected from Table 6 or Table 7 or its complement. In an embodiment, the kit further comprises information, in electronic or paper form, comprising instructions on how to determine if a subject is likely to be responsive to anti-cancer therapy. In an embodiment, the kit further comprises one or more control reference samples.

An aspect of the disclosure is a probe set for diagnosing and/or prognosing prostate cancer in a subject, the probe set comprising a plurality of probes for detecting a plurality of target nucleic acids, wherein the plurality of target nucleic acids comprises one or more gene sequences, or complements thereof, of genes selected from Table 6 or Table 7. In an embodiment, the at least one probe is detectably labeled. An aspect of the invention is a kit for detecting, diagnosing and/or prognosing prostate cancer comprising the probe set of claim. An aspect of the disclosure is a system for analyzing a prostate cancer to provide a diagnosis and/or prognosis to a subject having prostate cancer, the system comprising: a) the probe set of claim; and b) a computer model or algorithm for analyzing an expression level or expression profile of the plurality of target nucleic acids hybridized to the plurality of probes in a biological sample from a subject who has prostate cancer and determining that the patient does or does not have homologous recombination deficiency prostate cancer based on the nucleic acid expression levels. An aspect of the disclosure is a kit for diagnosing and/or prognosing prostate cancer in a subject comprising the system. In an embodiment, the kit further comprises a computer model or algorithm for designating a treatment modality for the subject. In an embodiment includes a computer model or algorithm for analyzing an expression level and/or expression profile of the target sequences in a sample. In some embodiments, the method further comprises a computer model or algorithm for correlating the expression level or expression profile with disease state or outcome. In other embodiments, the method further comprises a computer model or algorithm for designating a treatment modality for the individual. In yet other embodiments, the method further comprises a computer model or algorithm for normalizing expression level or expression profile of the target sequences. In some embodiments, the method further comprises sequencing the plurality of targets. In some embodiments, the method further comprises hybridizing the plurality of targets to a solid support. In some embodiments, the solid support is a bead or array. In some embodiments, assaying the expression level of a plurality of targets may comprise the use of a probe set. In some embodiments, assaying the expression level may comprise the use of a classifier. The classifier may comprise a probe selection region (PSR). In some embodiments, the classifier may comprise the use of an algorithm. The algorithm may comprise a machine learning algorithm. In some embodiments, assaying the expression level may also comprise sequencing the plurality of targets.

Further disclosed herein are classifiers for identifying homologous recombination deficiency prostate cancer, wherein the classifiers have an AUC value of at least about 0.40 to predict patient outcomes. In some embodiments, patient outcomes are selected from the group consisting of biochemical recurrence (BCR), metastasis (MET) and prostate cancer death (PCSM) after radical prostatectomy. The AUC of the classifier may be at least about 0.40, 0.45, 0.50, 0.55, 0.60, 0.61, 0.62, 0.63, 0.64, 0.65, 0.66, 0.67, 0.68, 0.69, 0.70 or more.

In any of the embodiments comprising identifying, determining, diagnosing and/or prognosing homologous recombination deficiency prostate cancer, comprising determining the level of expression or amplification of at least one or more genes of the disclosure, wherein the significance of the expression level of the one or more genes is based on one or more metrics selected from the group comprising T-test, P-value, KS (Kolmogorov Smirnov) P-value, accuracy, accuracy P-value, positive predictive value (PPV), negative predictive value (NPV), sensitivity, specificity, AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). The significance of the expression level of the one or more genes may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval) and Multivariable Analysis Hazard Ratio P-value (mvaHRPval). The genomic classifiers of the disclosure are useful for identifying homologous recombination deficiency prostate cancer and for predicting clinical characteristics of subjects with prostate cancer. In some embodiments, the clinical characteristics are selected from the group consisting of response to androgen deprivation therapy (ADP), time to metastatic recurrence, propensity for disease recurrence following surgery, fraction genome altered (FGA), luminal B tumor, seminal vesical invasion (SVI), lymph node invasion (LNI), prostate-specific antigen (PSA), and gleason score (GS).

In an embodiment of the method, kit, probe set or system disclosed above and herein, the plurality of targets comprise or consist of one or more targets selected from Table 6 or Table 7. In an embodiment of the method, kit, probe set or system disclosed above and herein, the plurality of targets comprise or consist of at least about 2, 3, 4, 5, 6, 7, 8, 9, 10, 11, 12, 13, 14, 15, 16, 17, 18, 19, 20, 25, 30, 35, 40, 45, or 50 targets selected from Table 6 or Table 7. In an embodiment of the method, kit, probe set or system disclosed above and herein, the plurality of targets comprise or consist of 2-10, 2-16, 8-16, 10-16, 13-16, 2-50, or 25-50 targets. In an embodiment of the method, kit, probe set or system disclosed above and herein, the plurality of targets comprise or consist of each of the targets from Table 6 and/or Table 7. In an embodiment of the method, kit, probe set or system disclosed above and herein, the plurality of targets comprise or consist of each of the targets from Table 7. In an embodiment of the method, kit, probe set or system disclosed above and herein, the plurality of targets comprise or consist of: GABRD, and TSEN15; GABRD, TSEN15, and DERL1; GABRD, TSEN15, DERL1, and TPT1; GABRD, TSEN15, DERL1, TPT1, and CCNB2; GABRD, TSEN15, DERL1, TPT1, CCNB2, and FDPS; GABRD, TSEN15, DERL1, TPT1, CCNB2, FDPS, and NUSAP1; GABRD, TSEN15, DERL1, TPT1, CCNB2, FDPS, NUSAP1, and HOXC4; GABRD, TSEN15, DERL1, TPT1, CCNB2, FDPS, NUSAP1, HOXC4, and ZNF185; GABRD, TSEN15, DERL1, TPT1, CCNB2, FDPS, NUSAP1, HOXC4, ZNF185, and METTL2A; GABRD, TSEN15, DERL1, TPT1, CCNB2, FDPS, NUSAP1, HOXC4, ZNF185, METTL2A, and ECHDC1; GABRD, TSEN15, DERL1, TPT1, CCNB2, FDPS, NUSAP1, HOXC4, ZNF185, METTL2A, ECHDC1, and ACTC1; GABRD, TSEN15, DERL1, TPT1, CCNB2, FDPS, NUSAP1, HOXC4, ZNF185, METTL2A, ECHDC1, ACTC1, and KCNN4; GABRD, TSEN15, DERL1, TPT1, CCNB2, FDPS, NUSAP1, HOXC4, ZNF185, METTL2A, ECHDC1, ACTC1, KCNN4, and ZNF69; GABRD, TSEN15, DERL1, TPT1, CCNB2, FDPS, NUSAP1, HOXC4, ZNF185, METTL2A, ECHDC1, ACTC1, KCNN4, ZNF69, and INSIG1; or GABRD, TSEN15, DERL1, TPT1, CCNB2, FDPS, NUSAP1, HOXC4, ZNF185, METTL2A, ECHDC1, ACTC1, KCNN4, ZNF69, INSIG1, and GJB2. In an embodiment of the method, kit, probe set or system disclosed above and herein, the sample is a biopsy. In an embodiment, the sample is a urine sample, a blood sample or a prostate tumor sample. In an embodiment, the blood sample is plasma, serum, or whole blood. In an embodiment, the subject is a human. In an embodiment, the measuring the level of expression comprises measuring the level of an RNA transcript.

### INCORPORATION BY REFERENCE

All publications, patents, and patent applications mentioned in this specification are herein incorporated by reference for the subject matter referenced herein and in their entireties to the same extent as if each individual publication, patent, or patent application was specifically and individually indicated to be incorporated by reference.

### BRIEF DESCRIPTION OF THE DRAWINGS

FIGS. 1A-E set forth data showing an embodiment of a transcriptomic model for homologous recombination deficiency using a mutational signature (HRDetect score), 82 tumors in TCGA were identified as HRD (a). A total of 113 genes were included in model building based on their specificity in prostate cancer (see methods and materials) and correlation with HRDetect score (b). The final HRD model had similar performance in TCGA training and validation cohorts (c) and better performance than a model previously developed for ovarian cancer (d-e). Abbreviations: HRD, homologous recombination deficiency; TCGA, The Cancer Genome Atlas; AUC, area under the curve.

FIGS. 2A-D set forth data showing an embodiment of genomic and clinical characteristics of HRDmodel tumors in TCGA based on the transcriptome, HRDmodel tumors were identified in 41% of TCGA tumors (a). HRDmodel tumors in TCGA were found to have higher values of fraction genome altered, higher MYC activity, lower p53 activity and lower predicted response to androgen deprivation therapy (b) HRDmodel was more predictive of genomic instability than single HR-gene mutations as assessed by fraction genome altered and the HRD score (pair-wise comparison p-values from Wilcoxon-rank sum; c). HRDmodel tumors tended to have more aggressive features and were enriched with luminal B subtype based on the PAM50 panel (d). Abbreviation: HR, homologous recombination.

FIGS. 3A-L set forth data showing an embodiment of clinical outcomes and molecular pathways in HRDmodel tumors. HRDmodel tumors were associated with shorter time to cancer recurrence following radical prostatectomy in three independent cohorts (a-c; p-values from log−rank) even after adjusting for relevant risk variables in Cox regression (d). A cohort of 7,000 tumors were used to assess differential expression of cancer hallmark pathways (e; p-values from Wilcoxon-rank sum) and of those pathways with −Log(FDR p-value) >250 or mean difference in expression >0.1 or <−0.1 (n=15; grey markers in e), three were also differentially expression in mCRPC (f; p-values from Wilcoxon−rank sum). In the mCRPC setting, the HRDmodel was more predictive of higher genomic instability as assessed by fraction genome altered than HR-gene mutations (g-h). In a cohort of 10 men with mCRPC who received the PARP inhibitor, olaparib, three were HRDmodel, each of whom experienced a PSA decline of ≥75% (i). The three men with HRDmodel tumors each experienced prolonged PSA progression-free survival. Abbreviations: TCGA, The Cancer Genome Atlas; JHMI, Johns Hopkins Medical Institute; PSA, prostate-specific antigen; HR, homologous recombination; AHR, adjusted hazard ratio; FDR, false discovery rate; mCRPC, metastatic castrate-resistant prostate cancer; PARP, Poly (ADP-ribose) polymerase; PSA, prostate-specific antigen.

FIG. 4 sets forth data showing an embodiment of gene ontology assessment performed for all the genes used for model building.

FIGS. 5A-B set forth data showing an embodiment of expression of heatmap of genes from an exemplary transcriptomic model for HRDmodel in GRID (a) and the metastatic castrate-resistant cohort (b).

### DETAILED DESCRIPTION OF THE INVENTION

The present disclosure discloses systems and methods for diagnosing, predicting, and/or identifying homologous recombination deficiency prostate cancer in a subject using expression-based analysis of a plurality of genes and/or targets disclosed herein. Generally, the method comprises (a) obtaining or having obtained a nucleic acid expression level for a plurality of genes and/or targets disclosed herein in a sample from a subject having prostate cancer; and (c) diagnosing, predicting and/or identifying homologous recombination deficiency prostate cancer based on the expression level of the plurality of genes, and optionally treating the subject with a particular treatment based on the determined HRD status as described herein.

Further disclosed herein are methods for identifying homologous recombination deficiency prostate cancer. Generally, the method comprises: (a) providing a sample comprising prostate cancer cells from a subject; (b) assaying the expression level for a plurality of genes and/or targets disclosed herein in the sample; and (c) determining that the patient has homologous recombination deficiency prostate cancer based on the expression level of the plurality of genes and/or targets, and optionally treating the subject with a particular treatment based on the determined HRD status as described herein.

Further disclosed herein are methods for prognosing homologous recombination deficiency prostate cancer. Generally, the method comprises: (a) obtaining or having obtained a nucleic acid expression level of a plurality of genes and/or targets disclosed herein, in a biological sample from a subject having prostate cancer; and b) prognosing the patient with homologous recombination deficiency prostate cancer based on the nucleic acid expression levels, and optionally treating the subject with a particular treatment based on the determined HRD status as described herein. In some instances, prognosing the prostate cancer comprises determining whether the cancer would respond to an anti-cancer therapy. In some embodiments, prognosing the prostate cancer comprises identifying the cancer as non-responsive to an anti-cancer therapy. Optionally, prognosing the prostate cancer comprises identifying the cancer as responsive to an anti-cancer therapy. In some embodiments, prognosing the prostate cancer comprises identifying the cancer as responsive to a PARP inhibitor.

Further disclosed herein are methods for treating homologous recombination deficiency prostate cancer. Generally, the method comprises: (a) obtaining or having obtained a nucleic acid expression level of a plurality of genes and/or targets disclosed herein, in a biological sample from a subject having prostate cancer; b) prognosing the patient with homologous recombination deficiency prostate cancer based on the nucleic acid expression levels; and c) administering an effective amount of a treatment to the patient based on the prognosis, wherein the treatment is an anti-cancer therapy as described herein, thereby treating the cancer.

The plurality of genes and/or targets disclosed herein comprise or consist of one or more genes/targets selected from Table 6 or Table 7. In some instances, the plurality of genes and/or targets disclosed herein consist of, comprises, comprises about, or comprises at least about 2, 3, 4, 5, 6, 7, 8, 9, 10, 11, 12, 13, 14, 15, 16, 17, 18, 19, 20, 25, 30, 35, 40, 45, or 50 genes/targets selected from Table 6 or Table 7, or a range of defined by any of the preceding values, for example 2-10, 2-16, 8-16, 10-16, 13-16, 2-50, or 25-50 genes/targets. In some instances, the plurality of genes and/or targets disclosed herein comprise or consist of each of the genes/targets from Table 6 and/or Table 7. In some instances, the plurality of genes and/or targets disclosed herein comprise or consist of each of the genes/targets from Table 7. In some instances, the plurality of genes and/or targets disclosed herein comprise or consist of:
GABRD, and TSEN15;
GABRD, TSEN115, and DERL1;
GABRD, TSEN15, DERL1, and TPT1;
GABRD, TSEN115, DERL1, TPT1, and CCNB2;
GABRD, TSEN15, DERL1, TPT1, CCNB2, and FDPS;
GABRD, TSEN15, DERL1, TPT1, CCNB2, FDPS, and NUSAP1;
GABRD, TSEN15, DERL1, TPT1, CCNB2, FDPS, NUSAP1, and HOXC4;
GABRD, TSEN15, DERL1, TPT1, CCNB2, FDPS, NUSAP1, HOXC4, and ZNF185;
GABRD, TSEN15, DERL1, TPT1, CCNB2, FDPS, NUSAP1, HOXC4, ZNF185, and METTL2A;
GABRD, TSEN15, DERL1, TPT1, CCNB2, FDPS, NUSAP1, HOXC4, ZNF185, METTL2A, and ECHDC1;
GABRD, TSEN15, DERL1, TPT1, CCNB2, FDPS, NUSAP1, HOXC4, ZNF185, METTL2A, ECHDC1, and ACTC1;
GABRD, TSEN15, DERL1, TPT1, CCNB2, FDPS, NUSAP1, HOXC4, ZNF185, METTL2A, ECHDC1, ACTC1, and KCNN4;
GABRD, TSEN15, DERL1, TPT1, CCNB2, FDPS, NUSAP1, HOXC4, ZNF185, METTL2A, ECHDC1, ACTC1, KCNN4, and ZNF69;
GABRD, TSEN15, DERL1, TPT1, CCNB2, FDPS, NUSAP1, HOXC4, ZNF185, METTL2A, ECHDC1, ACTC1, KCNN4, ZNF69, and INSIG1; or
GABRD, TSEN15, DERL1, TPT1, CCNB2, FDPS, NUSAP1, HOXC4, ZNF185, METTL2A, ECHDC1, ACTC1, KCNN4, ZNF69, INSIG1, and GJB2.

Assaying the expression level for a plurality of genes/targets in the sample may comprise applying the sample to a microarray. In some instances, assaying the expression level may comprise the use of an algorithm. The algorithm may be used to produce a classifier. In some embodiments, the classifier may comprise a probe selection region. In some instances, assaying the expression level for a plurality of genes/targets comprises detecting and/or quantifying the plurality of genes/targets. In some embodiments, assaying the expression level for a plurality of genes/targets comprises sequencing the plurality of genes/targets. In some embodiments, assaying the expression level for a plurality of genes/targets comprises amplifying the plurality of genes. In some embodiments, assaying the expression level for a plurality of genes/targets comprises quantifying the plurality of genes/targets. In some embodiments, assaying the expression level for a plurality of genes/targets comprises conducting a multiplexed reaction on the plurality of genes.

Before the present disclosure is described in further detail, it is to be understood that this disclosure is not limited to the particular methodology, compositions, articles or machines described, as such methods, compositions, articles or machines can, of course, vary. It is also to be understood that the terminology used herein is for the purpose of describing particular embodiments only, and is not intended to limit the scope of the present disclosure.

### Genes and Targets

The methods disclosed herein often comprise assaying the expression level of a plurality of genes and/or targets. The terms “target” and “gene” are used interchangeably throughout this disclosure with respect to the genes listed in Tables 6 and 7, and it is understood that either term can be and is used in reference to the genes listed in Tables 6 and 7. The plurality of genes and/or targets may comprise coding genes and/or targets and/or non-coding genes and/or targets of a protein-coding gene or a non protein-coding gene. A protein-coding gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non protein-coding gene structure may comprise an exon and intron. Usually, the exon region of a non protein-coding gene primarily contains a UTR. The non protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

In some instances, the plurality of genes and/or targets may be differentially expressed. In some instances, a plurality of probe selection regions (PSRs) is differentially expressed.

The plurality of genes and/or targets disclosed herein comprise or consist of one or more genes/targets selected from Table 6 or Table 7. In some instances, the plurality of genes and/or targets disclosed herein consist of, comprises, comprises about, or comprises at least about 2, 3, 4, 5, 6, 7, 8, 9, 10, 11, 12, 13, 14, 15, 16, 17, 18, 19, 20, 25, 30, 35, 40, 45, or 50 genes/targets selected from Table 6 or Table 7, or a range of defined by any of the preceding values, for example 2-10, 2-16, 8-16, 10-16, 13-16, 2-50, or 25-50 genes/targets. In some instances, the plurality of genes and/or targets disclosed herein comprise or consist of each of the genes/targets from Table 6 and/or Table 7. In some instances, the plurality of genes and/or targets disclosed herein comprise or consist of each of the genes/targets from Table 7. In some instances, the plurality of genes and/or targets disclosed herein comprise or consist of:
GABRD, and TSEN15;
GABRD, TSEN15, and DERL1;
GABRD, TSEN15, DERL1, and TPT1;
GABRD, TSEN15, DERL1, TPT1, and CCNB2;
GABRD, TSEN15, DERL1, TPT1, CCNB2, and FDPS;
GABRD, TSEN15, DERL1, TPT1, CCNB2, FDPS, and NUSAP1;
GABRD, TSEN15, DERL1, TPT1, CCNB2, FDPS, NUSAP1, and HOXC4;
GABRD, TSEN15, DERL1, TPT1, CCNB2, FDPS, NUSAP1, HOXC4, and ZNF185;
GABRD, TSEN15, DERL1, TPT1, CCNB2, FDPS, NUSAP1, HOXC4, ZNF185, and MET1L2A;
GABRD, TSEN15, DERL1, TPT1, CCNB2, FDPS, NUSAP1, HOXC4, ZNF185, METTL2A, and ECHDC1;
GABRD, TSEN15, DERL1, TPT1, CCNB2, FDPS, NUSAP1, HOXC4, ZNF185, METTL2A, ECHDC1, and ACTC1;
GABRD, TSEN15, DERL1, TPT1, CCNB2, FDPS, NUSAP1, HOXC4, ZNF185, METTL2A, ECHDC1, ACTC1, and KCNN4;
GABRD, TSEN15, DERL1, TPT1, CCNB2, FDPS, NUSAP1, HOXC4, ZNF185, METTL2A, ECHDC1, ACTC1, KCNN4, and ZNF69;
GABRD, TSEN15, DERL1, TPT1, CCNB2, FDPS, NUSAP1, HOXC4, ZNF185, METTL2A, ECHDC1, ACTC1, KCNN4, ZNF69, and INSIG1; or
GABRD, TSEN15, DERL1, TPT1, CCNB2, FDPS, NUSAP1, HOXC4, ZNF185, METTL2A, ECHDC1, ACTC1, KCNN4, ZNF69, INSIG1, and GJB2.

In some instances, the plurality of targets comprises a coding target, non-coding target, or any combination thereof. In some instances, the coding target comprises an exonic sequence. In other instances, the non-coding target comprises a non-exonic or exonic sequence. In some embodiments, a non-coding target comprises a UTR sequence, an intronic sequence, antisense, or a non-coding RNA transcript. In some instances, a non-coding target comprises sequences which partially overlap with a UTR sequence or an intronic sequence. A non-coding target also includes non-exonic and/or exonic transcripts. Exonic sequences may comprise regions on a protein-coding gene, such as an exon, UTR, or a portion thereof. Non-exonic sequences may comprise regions on a protein-coding, non protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In other instances, the plurality of targets comprises a non-coding RNA transcript.

The plurality of genes and/or targets may comprise one or more genes and/or targets selected from a classifier disclosed herein. The classifier may be generated from one or more models or algorithms. The one or more models or algorithms may be Naïve Bayes (NB), recursive Partitioning (Rpart), random forest (RF), support vector machine (SVM), k-nearest neighbor (KNN), high dimensional discriminate analysis (HDDA), or a combination thereof. The classifier may have an AUC of equal to or greater than 0.60. The classifier may have an AUC of equal to or greater than 0.61. The classifier may have an AUC of equal to or greater than 0.62. The classifier may have an AUC of equal to or greater than 0.63. The classifier may have an AUC of equal to or greater than 0.64. The classifier may have an AUC of equal to or greater than 0.65. The classifier may have an AUC of equal to or greater than 0.66. The classifier may have an AUC of equal to or greater than 0.67. The classifier may have an AUC of equal to or greater than 0.68. The classifier may have an AUC of equal to or greater than 0.69. The classifier may have an AUC of equal to or greater than 0.70. The classifier may have an AUC of equal to or greater than 0.75. The classifier may have an AUC of equal to or greater than 0.77. The classifier may have an AUC of equal to or greater than 0.78. The classifier may have an AUC of equal to or greater than 0.79. The classifier may have an AUC of equal to or greater than 0.80. The AUC may be clinically significant based on its 95% confidence interval (CI). The accuracy of the classifier may be at least about 70%. The accuracy of the classifier may be at least about 73%. The accuracy of the classifier may be at least about 75%. The accuracy of the classifier may be at least about 77%. The accuracy of the classifier may be at least about 80%. The accuracy of the classifier may be at least about 83%. The accuracy of the classifier may be at least about 84%. The accuracy of the classifier may be at least about 86%. The accuracy of the classifier may be at least about 88%. The accuracy of the classifier may be at least about 90%. The p-value of the classifier may be less than or equal to 0.05. The p-value of the classifier may be less than or equal to 0.04. The p-value of the classifier may be less than or equal to 0.03. The p-value of the classifier may be less than or equal to 0.02. The p-value of the classifier may be less than or equal to 0.01. The p-value of the classifier may be less than or equal to 0.008. The p-value of the classifier may be less than or equal to 0.006. The p-value of the classifier may be less than or equal to 0.004. The p-value of the classifier may be less than or equal to 0.002. The p-value of the classifier may be less than or equal to 0.001.

The plurality of genes and/or targets may comprise one or more genes and/or targets selected from a Random Forest (RF) classifier. The plurality of genes and/or targets may comprise two or more genes and/or targets selected from a Random Forest (RF) classifier. The plurality of genes and/or targets may comprise three or more genes and/or targets selected from a Random Forest (RF) classifier. The plurality of genes and/or targets may comprise 2, 3, 4, 5, 6, 7, 8, 9, 10, 11, 12, 13, 14, 15, 20, 25, 30, 40, 50 or more genes and/or targets selected from a Random Forest (RF) classifier. The RF classifier may be an RF2, and RF3, or an RF4 classifier. The RF classifier may be an RF50 classifier (e.g., a Random Forest classifier with 50 genes and/or targets).

A RF classifier of the present disclosure may comprise two or more genes and/or targets comprising two or more genes and/or targets selected from Table 6 or Table 7, or subgroups thereof as set forth herein (e.g., at ¶ [0030]).

The plurality of genes and/or targets may comprise one or more genes and/or targets selected from an SVM classifier. The plurality of genes and/or targets may comprise 2, 3, 4, 5, 6, 7, 8, 9, 10, 11, 12, 13, 14, 15, 20, 25, 30, 40, 50 or more genes and/or targets selected from an SVM classifier. The plurality of genes and/or targets may comprise 12, 13, 14, 15, 17, 20, 22, 25, 27, 30, 40, 50 or more genes and/or targets selected from an SVM classifier. The plurality of genes and/or targets may comprise 32, 35, 37, 40, 43, 45, 47, 50 or more genes and/or targets selected from an SVM classifier. The SVM classifier may be an SVM2 classifier.

A SVM classifier of the present disclosure may comprise two or more genes and/or targets comprising two or more genes and/or targets selected from Table 6 or Table 7, or subgroups thereof as set forth herein (e.g., at ¶ [0030]).

The plurality of genes and/or targets may comprise one or more genes and/or targets selected from a KNN classifier. The plurality of genes and/or targets may comprise 2, 3, 4, 5, 6, 7, 8, 9, 10 or more genes and/or targets selected from a KNN classifier. The plurality of genes and/or targets may comprise 12, 13, 14, 15, 17, 20, 22, 25, 27, 30 or more genes and/or targets selected from a KNN classifier. The plurality of genes and/or targets may comprise 32, 35, 37, 40, 43, 45, 47, 50 or more genes and/or targets selected from a KNN classifier.

The KNN classifier may be a KNN50 classifier. A KNN classifier of the present disclosure may comprise fifty or more genes and/or targets comprising fifty or more genes and/or targets selected from Table 6 or Table 7, or subgroups thereof as set forth herein (e.g., at ¶ [0030]).

The plurality of genes and/or targets may comprise one or more genes and/or targets selected from a Naïve Bayes (NB) classifier. The plurality of genes and/or targets may comprise 2, 3, 4, 5, 6, 7, 8, 9, 10 or more genes and/or targets selected from an NB classifier. The plurality of genes and/or targets may comprise 12, 13, 14, 15, 17, 20, 22, 25, 27, 30 or more genes and/or targets selected from an NB classifier. The plurality of genes and/or targets may comprise 32, 35, 37, 40, 43, 45, 47, 50 or more genes and/or targets selected from a NB classifier.

The NB classifier may be a NB2 classifier. An NB classifier of the present disclosure may comprise two or more genes and/or targets comprising two or more genes and/or targets selected from Table 6 or Table 7, or subgroups thereof as set forth herein (e.g., at ¶ [0030]).

The plurality of genes and/or targets may comprise one or more genes and/or targets selected from a recursive Partitioning (Rpart) classifier. The plurality of genes and/or targets may comprise 2, 3, 4, 5, 6, 7, 8, 9, 10 or more genes and/or targets selected from an Rpart classifier. The plurality of genes and/or targets may comprise 12, 13, 14, 15, 17, 20, 22, 25, 27, 30 or more genes and/or targets selected from an Rpart classifier. The plurality of genes and/or targets may comprise 32, 35, 37, 40, 43, 45, 47, 50 or more genes and/or targets selected from an Rpart classifier.

The Rpart classifier may be an Rpart2 classifier. An Rpart classifier of the present disclosure may comprise two or more genes and/or targets comprising two or more genes and/or targets selected from Table 6 or Table 7, or subgroups thereof as set forth herein (e.g., at ¶ [0030]).

The plurality of genes and/or targets may comprise one or more genes and/or targets selected from a high dimensional discriminate analysis (HDDA) classifier. The plurality of genes and/or targets may comprise two or more genes and/or targets selected from a high dimensional discriminate analysis (HDDA) classifier. The plurality of genes and/or targets may comprise three or more genes and/or targets selected from a high dimensional discriminate analysis (HDDA) classifier. The plurality of genes and/or targets may comprise 5, 6, 7, 8, 9, 10, 11, 12, 13, 14, 15, 20, 30, 40, 50 or more genes and/or targets selected from a high dimensional discriminate analysis (HDDA) classifier.

### Probes/Primers

The disclosure provides for a probe set for diagnosing, monitoring and/or identifying homologous recombination deficiency prostate cancer in a subject comprising a plurality of probes, wherein (i) the probes in the set are capable of detecting an expression level of at least one gene and/or target selected from Table 6 or Table 7, or subgroups thereof as set forth herein (e.g., at ¶ [0030]); and (ii) the expression level determines homologous recombination deficiency prostate cancer with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the disclosure, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from prostate cancer tissue samples and amplified, may yield a dataset that is then summarized and normalized by the fRMA technique. After removal (or filtration) of cross-hybridizing PSRs, and PSRs containing less than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

The polynucleotide probes of the disclosure may range in length from about 15 nucleotides to the full length of the coding target or non-coding target. In one embodiment of the disclosure, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. In some embodiments, the probe set comprises a combination of a coding target and non-coding target.

In other embodiments, the probe set comprise a plurality of target sequences that hybridize to at least about 5 coding targets and/or non-coding targets selected from Table 6 or Table 7, or subgroups thereof as set forth herein (e.g., at ¶ [0030]). In some embodiments, the probe set comprise a plurality of target sequences that hybridize to at least about 10 coding targets and/or non-coding targets selected from Table 6 or Table 7, or subgroups thereof as set forth herein (e.g., at ¶ [0030]). In some embodiments, the probe set comprise a plurality of target sequences that hybridize to at least about 15 coding targets and/or non-coding targets selected from Table 6 or Table 7, or subgroups thereof as set forth herein (e.g., at ¶ [0030]). In some embodiments, the probe set comprise a plurality of target sequences that hybridize to at least about 20 coding targets and/or non-coding targets selected from Table 6 or Table 7, or subgroups thereof as set forth herein (e.g., at ¶ [0030]). In some embodiments, the probe set comprise a plurality of target sequences that hybridize to at least about 30 coding targets and/or non-coding targets selected from Table 6 or Table 7, or subgroups thereof as set forth herein (e.g., at ¶ [0030]). In some embodiments, the probe set comprise a plurality of target sequences that hybridize to at least about 40 coding targets and/or non-coding targets selected from Table 6 or Table 7, or subgroups thereof as set forth herein (e.g., at ¶ [0030]). In some embodiments, the probe set comprise a plurality of target sequences that hybridize to at least about 50 coding targets and/or non-coding targets selected from Table 6 or Table 7, or subgroups thereof as set forth herein (e.g., at ¶ [0030]).

The system of the present disclosure further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. Generally, the exact composition of the primer sequences is not critical to the disclosure, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. In some embodiments, these primers may be used in combination with probes, such as molecular beacons in amplifications using real-time PCR

In one embodiment, the primers or primer pairs, when used in an amplification reaction, specifically amplify at least a portion of a nucleic acid sequence of a target selected Table 6 or Table 7, or subgroups thereof as set forth herein (e.g., at ¶ [0030]), an RNA form thereof, or a complement to either thereof.

A label can optionally be attached to or incorporated into a probe or primer polynucleotide to allow detection and/or quantitation of a target polynucleotide representing the target sequence of interest. The target polynucleoide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the disclosure described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

In some embodiments, polynucleotides of the disclosure comprise at least 20 consecutive bases of the nucleic acid sequence of a target selected from Table 6 or Table 7, or subgroups thereof as set forth herein (e.g., at ¶ [0030]) or a complement thereto. The polynucleotides may comprise at least 21, 22, 23, 24, 25, 27, 30, 32, 35 or more consecutive bases of the nucleic acids sequence of a target selected from Table 6 or Table 7, or subgroups thereof as set forth herein (e.g., at ¶ [0030]), as applicable.

The polynucleotides may be provided in a variety of formats, including as solids, in solution, or in an array. The polynucleotides may optionally comprise one or more labels, which may be chemically and/or enzymatically incorporated into the polynucleotide.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

### Diagnostic Samples

Diagnostic samples for use with the systems and in the methods of the present disclosure comprise nucleic acids suitable for providing RNAs expression information. In principle, the biological sample from which the expressed RNA is obtained and analyzed for target sequence expression can be any material suspected of comprising prostate cancer tissue or cells. The diagnostic sample can be a biological sample used directly in a method of the disclosure. In some embodiments, the diagnostic sample can be a sample prepared from a biological sample.

In one embodiment, the sample or portion of the sample comprising or suspected of comprising cancer tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from urine. In some embodiments, the sample is from blood, plasma or serum. In some embodiments, the sample is from saliva.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences, Hatfield, PA). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

### RNA Extraction

RNA can be extracted and purified from biological samples using any suitable technique. A number of techniques are known in the art, and several are commercially available (e.g., FormaPure nucleic acid extraction kit, Agencourt Biosciences, Beverly MA, High Pure FFPE RNA Micro Kit, Roche Applied Science, Indianapolis, IN). RNA can be extracted from frozen tissue sections using TRIzol (Invitrogen, Carlsbad, CA) and purified using RNeasy Protect kit (Qiagen, Valencia, CA). RNA can be further purified using DNAse I treatment (Ambion, Austin, TX) to eliminate any contaminating DNA. RNA concentrations can be made using a Nanodrop ND-1000 spectrophotometer (Nanodrop Technologies, Rockland, DE). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, CA).

### Kits

Kits for performing the desired method(s) are also provided, and comprise a container or housing for holding the components of the kit, one or more vessels containing one or more nucleic acid(s), and optionally one or more vessels containing one or more reagents. The reagents include those described in the composition of matter section above and elsewhere herein, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for those subsets and combinations of target sequences described herein. The primers or pairs of primers suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100 or more primers or pairs of primers suitable for selectively amplifying one or more targets selected from Table 6 or Table 7, or subgroups thereof as set forth herein (e.g., at ¶ [0030]).

In some embodiments, the primers or primer pairs of the kit, when used in an amplification reaction, specifically amplify a non-coding target, coding target, exonic, or non-exonic target described herein, a nucleic acid sequence corresponding to a target selected from Table 6 or Table 7, or subgroups thereof as set forth herein (e.g., at ¶ [0030]), an RNA form thereof, or a complement to either thereof. The kit may include a plurality of such primers or primer pairs which can specifically amplify a corresponding plurality of different amplify a non-coding target, coding target, exonic, or non-exonic transcript described herein, a nucleic acid sequence corresponding to a target selected from Table 6 or Table 7, or subgroups thereof as set forth herein (e.g., at ¶ [0030]), RNA forms thereof, or complements thereto. At least two, three, four or five primers or pairs of primers suitable for selectively amplifying the one or more targets can be provided in kit form. In some embodiments, the kit comprises from five to fifty primers or pairs of primers suitable for amplifying the one or more targets.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from patients showing no evidence of disease, as well as tissue and/or nucleic acids obtained from or representative of tumor samples from patients that develop systemic cancer.

The nucleic acids may be provided in an array format, and thus an array or microarray may be included in the kit. The kit optionally may be certified by a government agency for use in prognosing the disease outcome of cancer patients and/or for designating a treatment modality.

Instructions for using the kit to perform one or more methods of the disclosure can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

### Amplification and Hybridization

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

The term “amplification” has it plain and ordinary meaning to one of skill in the art read view of the present specification, and means any process of producing at least one copy of a nucleic acid, for example an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self-sustained sequence replication (3SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coil, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Tfi, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, CA), including the WT-Ovation™ System, WT-Ovation™ System v2, WT-Ovation™ Pico System, WT-Ovation™ FFPE Exon Module, WT-Ovation™ FFPE Exon Module RiboAmp and RiboAmp^{Plus} RNA Amplification Kits (MDS Analytical Technologies (formerly Arcturus) (Mountain View, CA), Genisphere, Inc. (Hatfield, PA), including the RampUp Plus™ and SenseAmp™ RNA Amplification kits, alone or in combination. Amplified nucleic acids may be subjected to one or more purification reactions after amplification and labeling, for example using magnetic beads (e.g., RNAClean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, CA), SmartCycler® 9600 or GeneXpert® Systems (Cepheid, Sunnyvale, CA), ABI 7900 HT Fast Real Time PCR system (Applied Biosystems, Foster City, CA), LightCycler® 480 System (Roche Molecular Systems, Pleasanton, CA), xMAP 100 System (Luminex, Austin, TX) Solexa Genome Analysis System (Illumina, Hayward, CA), OpenArray Real Time qPCR (BioTrove, Woburn, MA) and BeadXpress System (Illumina, Hayward, CA).

Detection and/or Quantification of Target Sequences

Any method of detecting and/or quantitating the expression of the encoded target sequences can in principle be used in the disclosure. The expressed target sequences can be directly detected and/or quantitated, or may be copied and/or amplified to allow detection of amplified copies of the expressed target sequences or its complement.

Methods for detecting and/or quantifying a target can include Northern blotting, sequencing, array or microarray hybridization, by enzymatic cleavage of specific structures (e.g., an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis.

In some instances, target sequences may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target include single-molecule sequencing (e.g., Helicos, PacBio), sequencing by synthesis (e.g., Illumina, Ion Torrent), sequencing by ligation (e.g., ABI SOLiD), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

### Reverse Transcription for QRT-PCR Analysis

Reverse transcription can be performed by any method known in the art. For example, reverse transcription may be performed using the Omniscript kit (Qiagen, Valencia, CA), Superscript III kit (Invitrogen, Carlsbad, CA), for RT-PCR Target-specific priming can be performed in order to increase the sensitivity of detection of target sequences and generate target-specific cDNA.

### TaqMan® Gene Expression Analysis

TaqMan®RT-PCR can be performed using Applied Biosystems Prism (ABI) 7900 HT instruments in a 5 1.11 volume with target sequence-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

### Classification Arrays

The present disclosure contemplates that a probe set or probes derived therefrom may be provided in an array format. In the context of the present disclosure, an “array” is a spatially or logically organized collection of polynucleotide probes. An array comprising probes specific for a coding target, non-coding target, or a combination thereof may be used. In some embodiments, an array comprising probes specific for two or more of transcripts of a target selected from Table 6 or Table 7, or subgroups thereof as set forth herein (e.g., at ¶ [0030]), or a product derived thereof can be used. Desirably, an array may be specific for 5, 10, 15, 16, 20, 25, 30, 40, 50 or more of transcripts of a target selected from Table 6 or Table 7, or subgroups thereof as set forth herein (e.g., at ¶ [0030]). Expression of these sequences may be detected alone or in combination with other transcripts. In some embodiments, an array is used which comprises a wide range of sensor probes for prostate-specific expression products, along with appropriate control sequences. In some instances, the array may comprise the Human Exon 1.0 ST Array (HuEx 1.0 ST, Affymetrix, Inc., Santa Clara, CA.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment of the present disclosure, the Cancer Prognosticarray is a chip.

### Data Analysis

In some embodiments, one or more pattern recognition methods can be used in analyzing the expression level of target sequences and/or genes. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score indicates the probability that a biological sample is from a patient who may exhibit no evidence of disease, who may exhibit systemic cancer, or who may exhibit biochemical recurrence. The likelihood score can be used to distinguish these disease states. The models and/or algorithms can be provided in machine readable format, and may be used to correlate expression levels or an expression profile with a disease state, and/or to designate a treatment modality for a patient or class of patients.

Assaying the expression level for a plurality of targets may comprise the use of an algorithm or classifier. Array data can be managed, classified, and analyzed using techniques known in the art. Assaying the expression level for a plurality of targets may comprise probe set modeling and data pre-processing. Probe set modeling and data pre-processing can be derived using the Robust Multi-Array (RMA) algorithm or variants GC-RMA, fRMA, Probe Logarithmic Intensity Error (PLIER) algorithm or variant iterPLIER. Variance or intensity filters can be applied to pre-process data using the RMA algorithm, for example by removing target sequences with a standard deviation of <10 or a mean intensity of <100 intensity units of a normalized data range, respectively.

In some embodiments, assaying the expression level for a plurality of targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). In some embodiments, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. In some embodiments, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. In some embodiments, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Cancer

The systems, compositions and methods disclosed herein may be used to diagnosis, monitor and/or predict the status or outcome of a cancer. In some embodiments, the classifiers and methods disclosed herein are used to identify homologous recombination deficiency prostate cancer. Generally, a cancer is characterized by the uncontrolled growth of abnormal cells anywhere in a body. The abnormal cells may be termed cancer cells, malignant cells, or tumor cells. Cancer is not confined to humans, animals and other living organisms can get cancer.

In some instances, the cancer may be a recurrent and/or refractory cancer. Most cancers can be classified as a carcinoma, sarcoma, leukemia, lymphoma, myeloma, or a central nervous system cancer.

The cancer may be a sarcoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

In some embodiments, the cancer may be a carcinoma. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis. Preferably, the cancer is a prostate cancer. In some embodiments, the cancer may be a thyroid cancer, bladder cancer, or pancreatic cancer.

In some embodiments, the cancer is ovarian cancer. Ovarian cancer occurs when abnormal cells in the ovary begin to multiply out of control and form a tumor. If left untreated, the tumor can spread to other parts of the body. This is called metastatic ovarian cancer. Ovarian cancer often goes undetected until it has spread within the pelvis and belly. At this late stage, ovarian cancer is more difficult to treat and can be fatal.

In some instances, the cancer is a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavity (pleura) or lining of the abdomen (peritoneum). The mesothelioma may be due to asbestos exposure. The cancer may be a brain cancer, such as a glioblastoma.

In some embodiments, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The cancer may be a leukemia. The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic-leukemia.

In some instances, the cancer is a lymphoma. Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

### Cancer Staging

Diagnosing, predicting, or monitoring a status or outcome of a cancer may comprise determining the stage of the cancer. Generally, the stage of a cancer is a description (usually numbers I to IV with IV having more progression) of the extent the cancer has spread. The stage often takes into account the size of a tumor, how deeply it has penetrated, whether it has invaded adjacent organs, how many lymph nodes it has metastasized to (if any), and whether it has spread to distant organs. Staging of cancer can be used as a predictor of survival, and cancer treatment may be determined by staging. Determining the stage of the cancer may occur before, during, or after treatment. The stage of the cancer may also be determined at the time of diagnosis.

Cancer staging can be divided into a clinical stage and a pathologic stage. Cancer staging may comprise the TNM classification. Generally, the TNM Classification of Malignant Tumours (TNM) is a cancer staging system that describes the extent of cancer in a patient's body. T may describe the size of the tumor and whether it has invaded nearby tissue, N may describe regional lymph nodes that are involved, and M may describe distant metastasis (spread of cancer from one body part to another). In the TNM (Tumor, Node, Metastasis) system, clinical stage and pathologic stage are denoted by a small “c” or “p” before the stage (e.g., cT3N1MO or pT2N0).

Often, clinical stage and pathologic stage may differ. Clinical stage may be based on all of the available information obtained before a surgery to remove the tumor. Thus, it may include information about the tumor obtained by physical examination, radiologic examination, and endoscopy. Pathologic stage can add additional information gained by examination of the tumor microscopically by a pathologist. Pathologic staging can allow direct examination of the tumor and its spread, contrasted with clinical staging which may be limited by the fact that the information is obtained by making indirect observations at a tumor which is still in the body. The TNM staging system can be used for most forms of cancer.

In some embodiments, staging may comprise Ann Arbor staging. Generally, Ann Arbor staging is the staging system for lymphomas, both in Hodgkin's lymphoma (previously called Hodgkin's disease) and Non-Hodgkin lymphoma (abbreviated NHL). The stage may depend on both the place where the malignant tissue is located (as located with biopsy, CT scanning and increasingly positron emission tomography) and on systemic symptoms due to the lymphoma (“B symptoms”: night sweats, weight loss of >10% or fevers). The principal stage may be determined by location of the tumor. Stage I may indicate that the cancer is located in a single region, usually one lymph node and the surrounding area. Stage I often may not have outward symptoms. Stage II can indicate that the cancer is located in two separate regions, an affected lymph node or organ and a second affected area, and that both affected areas are confined to one side of the diaphragm—that is, both are above the diaphragm, or both are below the diaphragm. Stage III often indicates that the cancer has spread to both sides of the diaphragm, including one organ or area near the lymph nodes or the spleen. Stage IV may indicate diffuse or disseminated involvement of one or more extralymphatic organs, including any involvement of the liver, bone marrow, or nodular involvement of the lungs.

Modifiers may also be appended to some stages. For example, the letters A, B, E, X, or S can be appended to some stages. Generally, A or B may indicate the absence of constitutional (B-type) symptoms is denoted by adding an “A” to the stage; the presence is denoted by adding a “B” to the stage. E can be used if the disease is “extranodal” (not in the lymph nodes) or has spread from lymph nodes to adjacent tissue. X is often used if the largest deposit is >10 cm large (“bulky disease”), or whether the mediastinum is wider than ⅓ of the chest on a chest X-ray. S may be used if the disease has spread to the spleen.

The nature of the staging may be expressed with CS or PS. CS may denote that the clinical stage as obtained by doctor's examinations and tests. PS may denote that the pathological stage as obtained by exploratory laparotomy (surgery performed through an abdominal incision) with splenectomy (surgical removal of the spleen).

### Homologous Recombination Deficiency Cancer

The inventors discovered that genomic classifiers for identifying homologous recombination deficient prostate cancer is prognostic and predicts response to anti-cancer therapies, including, for example, PARP inhibitors. Homologous recombination (HR) comprises a series of interrelated pathways that function in the repair of DNA double-stranded breaks (DSBs) and interstrand crosslinks (ICLs). In addition, recombination provides critical support for DNA replication in the recovery of stalled or broken replication forks, contributing to tolerance of DNA damage. A central core of proteins, most critically the RecA homolog Rad51, catalyzes the key reactions that typify HR homology search and DNA strand invasion. The diverse functions of recombination are reflected in the need for context-specific factors that perform supplemental functions in conjunction with the core proteins. The inability to properly repair complex DNA damage and resolve DNA replication stress leads to genomic instability and contributes to cancer etiology. Mutations in the BRCA2 recombination gene cause predisposition to breast and ovarian cancer as well as Fanconi anemia, a cancer predisposition syndrome characterized by a defect in the repair of DNA interstrand crosslinks. The cellular functions of recombination are also germane to DNA-based treatment modalities of cancer, which target replicating cells by the direct or indirect induction of DNA lesions that are substrates for recombination pathways.

Inactivating mutations in the genes responsible for DNA repair pathways render tumors homologous recombination deficient (HRD) that lead to sensitivity to PARP inhibitors, a novel class of cancer therapy. Because tumors deficient in HRD are sensitive to PARP inhibitors, the consequence of not identifying tumors that harbor HRD is that treatment with these agents may be delayed or not given.

Differential expression analysis one or more of the genes and/or targets listed in Table 6 or Table 7, or subgroups thereof as set forth herein (e.g., at ¶ [0030]), allow for the identification of homologous recombination deficient prostate cancer. Genomic classifiers of the disclosure may be used to predict outcomes such as distant metastasis-free survival (DMFS), biochemical recurrence-free survival (bRFS), prostate cancer specific survival (PCSS), and overall survival (OS).

### Therapeutic Regimens

Diagnosing, predicting, or monitoring a status or outcome of a cancer may comprise treating a cancer or preventing a cancer progression. In addition, diagnosing, predicting, or monitoring a status or outcome of a cancer may comprise identifying or predicting responders to an anti-cancer therapy. In some instances, diagnosing, predicting, or monitoring may comprise determining a therapeutic regimen. Determining a therapeutic regimen may comprise administering an anti-cancer therapy, such as, for example, a PARP inhibitor. In some embodiments, determining a therapeutic regimen may comprise modifying, recommending, continuing or discontinuing an anti-cancer regimen. In some instances, if the sample expression patterns are consistent with the expression pattern for a known disease or disease outcome, the expression patterns can be used to designate one or more treatment modalities (e.g., therapeutic regimens, anti-cancer regimen). An anti-cancer regimen may comprise one or more anti-cancer therapies. Examples of anti-cancer therapies include surgery, chemotherapy, radiation therapy, immunotherapy/biological therapy, photodynamic therapy.

PARP inhibitors are a group of pharmacological inhibitors of the enzyme poly ADP ribose polymerase (PARP). They are developed for multiple indications, including the treatment of heritable cancers. Several forms of cancer are more dependent on PARP than regular cells, making PARP (PARP1, PARP2) an attractive target for cancer therapy. PARP inhibitors appear to improve progression-free survival in women with recurrent platinum-sensitive ovarian cancer. Drugs that inhibit PARP1 cause multiple double strand breaks to form in this way, and in tumors with BRCA1, BRCA2 or PALB2 mutations, these double strand breaks cannot be efficiently repaired, leading to the death of the cells. Normal cells that don't replicate their DNA as often as cancer cells, and that lack any mutated BRCA1 or BRCA2 still have homologous repair operating, which allows them to survive the inhibition of PARP.

PARP inhibitors lead to trapping of PARP proteins on DNA in addition to blocking their catalytic action. This interferes with replication, causing cell death preferentially in cancer cells, which grow faster than non-cancerous cells. Examples of PARP inhibitors include, Olaparib, Rucaparib, Niraparib, Talazoparib, 3-Aminobenzamide, Veliparib, Pamiparib, CEP 9722, and E7016.

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MR may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. In some embodiments, alkylating agents may chemically modify a cell's DNA.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin. Podophyllotoxin is a plant-derived compound that may help with digestion and may be used to produce cytostatic drugs such as etoposide and teniposide. They may prevent the cell from entering the GI phase (the start of DNA replication) and the replication of DNA (the S phase).

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type H inhibitors include amsacrine, etoposide, etoposide phosphate, and teniposide.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antibiotics include, but are not limited to, actinomycin, anthracyclines, doxorubicin, daunorubicin, valrubicin, idarubicin, epirubicin, bleomycin, plicamycin, and mitomycin.

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therapy (IGRT). In IGRT, repeated imaging scans (CT, MRI, or PET) may be performed during treatment. These imaging scans may be processed by computers to identify changes in a tumor's size and location due to treatment and to allow the position of the patient or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the patient in the same manner as a normal CT scanner. Tomotherapy machines can capture CT images of the patient's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and patient positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, patients may receive radiation therapy to the entire brain (called whole-brain radiation therapy) in addition to SRS. SRS requires the use of a head frame or other device to immobilize the patient during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a prostate tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in patients using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Bracytherapy may be used to treat patients with breast cancer who have undergone breast-conserving surgery.

The placement of brachytherapy sources can be temporary or permanent. For permanent brachytherapy, the sources may be surgically sealed within the body and left there, even after all of the radiation has been given off. In some instances, the remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a patient may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxar®) may be used for the treatment of certain types of cancer, such as NHL. In this regimen, nonradioactive tositumomab antibodies may be given to patients first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and prostate cancers.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit patients undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, esophagus, breast, uterus, ovary, prostate, kidney, colon, and rectum.

Another type of biological therapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a patient's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as colorectal, ovarian, and prostate.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in patients with tumors that produce excess amounts of a protein called HER2. In some embodiments, MOABs may be used to treat lymphoma, leukemia, melanoma, and cancers of the brain, breast, lung, kidney, colon, rectum, ovary, prostate, and other areas.

Cancer vaccines are another form of biological therapy. Cancer vaccines may be designed to encourage the patient's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, colon, and rectum. Cancer vaccines can be used in combination with other anti-cancer therapies.

Gene therapy is another example of a biological therapy. Gene therapy may involve introducing genetic material into a person's cells to fight disease. Gene therapy methods may improve a patient's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outpatient procedure. PDT may also be repeated and may be used with other therapies, such as surgery, radiation, or chemotherapy.

Extracorporeal photopheresis (ECP) is a type of PDT in which a machine may be used to collect the patient's blood cells. The patient's blood cells may be treated outside the body with a photosensitizing agent, exposed to light, and then returned to the patient. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in patients for whom the usual treatments are not appropriate, and to relieve symptoms in patients with non-small cell lung cancer that obstructs the airways. Porfimer sodium may also be used for the treatment of precancerous lesions in patients with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a patient's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO2) lasers, argon lasers, and neodymium:yttrium-aluminum-garnet (Nd:YAG) lasers. Each of these can shrink or destroy tumors and can be used with endoscopes. CO2 and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

For patients with high test scores consistent with systemic disease outcome after prostatectomy, additional treatment modalities such as adjuvant chemotherapy (e.g., docetaxel, mitoxantrone and prednisone), systemic radiation therapy (e.g., samarium or strontium) and/or anti-androgen therapy (e.g., surgical castration, finasteride, dutasteride) can be designated. Such patients would likely be treated immediately with anti-androgen therapy alone or in combination with radiation therapy in order to eliminate presumed micro-metastatic disease, which cannot be detected clinically but can be revealed by the target sequence expression signature.

Such patients can also be more closely monitored for signs of disease progression. For patients with intermediate test scores consistent with biochemical recurrence only (BCR-only or elevated PSA that does not rapidly become manifested as systemic disease only localized adjuvant therapy (e.g., radiation therapy of the prostate bed) or short course of anti-androgen therapy would likely be administered. For patients with low scores or scores consistent with no evidence of disease (NED) adjuvant therapy would not likely be recommended by their physicians in order to avoid treatment-related side effects such as metabolic syndrome (e.g., hypertension, diabetes and/or weight gain), osteoporosis, proctitis, incontinence or impotence. Patients with samples consistent with NED could be designated for watchful waiting, or for no treatment. Patients with test scores that do not correlate with systemic disease but who have successive PSA increases could be designated for watchful waiting, increased monitoring, or lower dose or shorter duration anti-androgen therapy.

Target sequences can be grouped so that information obtained about the set of target sequences in the group can be used to make or assist in making a clinically relevant judgment such as a diagnosis, prognosis, or treatment choice.

A patient report is also provided comprising a representation of measured expression levels of a plurality of target sequences in a biological sample from the patient, wherein the representation comprises expression levels of target sequences corresponding to any one, two, three, four, five, six, eight, ten, twenty, thirty, forty, fifty or more of the target sequences corresponding to a target selected from Table 6 or Table 7, the subgroups described herein (e.g., at ¶ [0030]), or a combination thereof. In some embodiments, the representation of the measured expression level(s) may take the form of a linear or nonlinear combination of expression levels of the target sequences of interest. The patient report may be provided in a machine (e.g., a computer) readable format and/or in a hard (paper) copy. The report can also include standard measurements of expression levels of said plurality of target sequences from one or more sets of patients with known disease status and/or outcome. The report can be used to inform the patient and/or treating physician of the expression levels of the expressed target sequences, the likely medical diagnosis and/or implications, and optionally may recommend a treatment modality for the patient.

Also provided are representations of the gene expression profiles useful for treating, diagnosing, prognosticating, and otherwise assessing disease. In some embodiments, these profile representations are reduced to a medium that can be automatically read by a machine such as computer readable media (magnetic, optical, and the like). The articles can also include instructions for assessing the gene expression profiles in such media. For example, the articles may comprise a readable storage form having computer instructions for comparing gene expression profiles of the portfolios of genes described above and elsewhere herein. The articles may also have gene expression profiles digitally recorded therein so that they may be compared with gene expression data from patient samples. In some embodiments, the profiles can be recorded in different representational format. A graphical recordation is one such format. Clustering algorithms can assist in the visualization of such data.

### Clinical Associations and Patient Outcomes

Genomic classifiers for identifying HRD cancers of the disclosure have distinct clinical associations. Clinical associations that correlate to such genomic classifiers include, for example, preoperative serum PSA, fraction genome altered (FGA), Gleason score (GS), extraprostatic extension (EPE), surgical margin status (SM), lymph node involvement (LNI), and seminal vesicle invasion (SVI). In some embodiments, the genomic classifiers of the disclosure are used to predict patient outcomes such as biochemical recurrence (BCR), metastasis (MET) and prostate cancer death (PCSM) after radical prostatectomy. In other embodiments, genomic classifiers of the disclosure are used to predict patient outcomes such as distant metastasis-free survival (DMFS), biochemical recurrence-free survival (bRFS), prostate cancer specific survival (PCSS), and overall survival (OS).

### Treatment Response Prediction

In some embodiments, the genomic classifiers of the disclosure are useful for predicting response to anticancer therapy (e.g., PARP inhibitors) following radical prostatectomy (see Example 1). Additionally, the genomic classifiers are useful for predicting response to androgen deprivation therapy. Androgen deprivation therapy (ADT), also called androgen suppression therapy, is an antihormone therapy whose main use is in treating prostate cancer. Prostate cancer cells usually require androgen hormones, such as testosterone, to grow. ADT reduces the levels of androgen hormones, with drugs or surgery, to prevent the prostate cancer cells from growing. The pharmaceutical approaches include antiandrogens and chemical castration.

### Examples

### Example 1: A Genetic Classifier to Identify Homologous Recombination Deficiency in Prostate Cancer and Predict Response to Therapy

A genetic signature to identify homologous recombination deficiency in prostate cancer tissue and predict response to therapy was developed as follows. Data on tumors derived from four radical prostatectomy (RP) cohorts and one metastasis biopsy cohort was included in this study. Tumors from Mayo Clinic (n=235) were derived from patients who underwent RP between 2000 and 2006 and previously served as a validation cohort for a genomic classifier. Tumors from JHMI were comprised of two groups: 1) Consisted of 355 intermediate- or high-risk patients treated with RP between 1995 and 2005 2) Consisted 143 Black men treated from 2006 to 2010. The Decipher Genomic Resource Information Database (GRID; NCT02609269; n=7000) is a large prospective registry of tumors from the clinical use of the Decipher RP test from December 2015 through September 2017.

Expression profiling for tumors from Mayo Clinic, JHMI, and the GRID were performed in a Clinical Laboratory Improvement Amendments (CLIA)-certified laboratory facility (Decipher Biosciences, San Diego, CA, USA). All tumors underwent central pathology review to assure at least 0.5 mm² of tumor with ≥60% tumor cellularity were required for sampling. RNA extraction and purification were performed using RNeasy FFPE (Qiagen, Valencia, CA). Ovation WTA FFPE system (NuGen, San Carlos, CA) was used for amplification and labeling of RNA, which was then hybridized to Human Exon 1.0 ST GeneChips (Thermo-Fisher, Carlsbad, CA) and quality control was performed using Affymetrix Power Tools. Single Channel Array Normalization algorithm was used for normalization.

Information regarding collection and processing of samples for TCGA (n=491) and a cohort of biopsies from men with metastatic castrate-resistant prostate cancer (mCRPC; n=118) have been previously described. Data for these cohorts were publicly available and downloaded from cBioPortal which included patient and tumor characteristics and both transcriptomic and genomic profiling (https://www.cbioportal.org/). Data from the Michigan Oncology Sequencing Project (MI-ONCOSEQ) prospective observation series were queried to identify patients treated with olaparib that had undergone whole exome sequencing and RNAseq. Ten patients with mCRPC were identified. Expression data from these three cohorts were quantile matched to the GRID such that all expression models and signatures could be directly compared. Patient and tumor characteristics for each cohort are shown below in Tables 1-5.

TABLE 1
The cancer genome atlas cohort

| Characteristic | n (%) |
|---|---|
| All | 491 (100) |
| Age, years | |
| Mean (SD) | 61.0 (6.8) |
| Grade group | |
| 1 | 45 (9.2) |
| 2 | 145 (29.5) |
| 3 | 99 (20.2) |
| 4 | 63 (12.8) |
| 5 | 139 (28.3) |
| PSA, ng/mL | |
| Mean (SD) | 11.0 (12.3) |
| Organ confined | |
| Yes | 186 (37.9) |
| No | 298 (60.7) |
| Surgical margin positive | |
| No | 327 (66.6) |
| Yes | 149 (30.3) |
| Unknown | 15 (3.1) |
| Disease recurrence | |
| No | 396 (80.7) |
| Yes | 89 (18.1) |
| Unknown | 6 (1.2) |
| Follow up, months | |
| Median (IQR) | 28.2 (15.8-165.1) |

TABLE 2
Mayo clinic cohort

| Characteristic | n (%) |
|---|---|
| All | 235 (100) |
| Age, years | |
| Mean (SD) | 63.1 (7.4) |
| Grade group | |
| 1 | 17 (7.2) |
| 2 | 78 (33.2) |
| 3 | 41 (17.4) |
| 4 | 39 (16.6) |
| 5 | 59 (25.1) |
| Unknown | 1 (0.4) |
| PSA, ng/mL | |
| Mean (SD) | 14.7 (17.7) |
| Organ confined | |
| Yes | 97 (41.3) |
| No | 138 (58.7) |
| Metastatic recurrence | |
| No | 159 (67.7) |
| Yes | 76 (32.3) |
| Follow up, years | |
| Median (IQR) | 7 (5-9) |

TABLE 3
Johns Hopkins Medical Institute cohort

| Characteristic | n (%) |
|---|---|
| All | 498 (100) |
| Age, years | |
| Mean (SD) | 59.0 (6.3) |
| Grade group | |
| 1 | 30 (6) |
| 2 | 174 (34.9) |
| 3 | 123 (24.7) |
| 4 | 59 (11.8) |
| 5 | 112 (22.5) |
| PSA, ng/mL | |
| Mean (SD) | 10.5 (8.1) |
| Organ confined | |
| Yes | 342 (68.7) |
| No | 150 (30.1) |
| Unknown | 6 (1.2) |
| Surgical margin positive | |
| No | 348 (69.9) |
| Yes | 147 (29.5) |
| Unknown | 3 (0.6) |
| Metastatic recurrence | |
| No | 356 (71.5) |
| Yes | 142 (28.5) |
| Follow up, years | |
| Median (IQR) | 8 (5-12) |

TABLE 4
GRID cohort

| Characteristic | n (%) |
|---|---|
| All | n = 7000 |
| Age, years | |
| Mean (SD) | 63.8 (40.6) |
| Grade group | |
| 1 | 382 (5.5) |
| 2 | 2156 (30.8) |
| 3 | 1391 (19.9) |
| 4 | 446 (6.4) |
| 5 | 621 (8.9) |
| Unknown | 2004 (28.6) |
| PSA, ng/mL | |
| Mean (SD) | 9.4 (15.2) |
| Organ confined | |
| Yes | 2115 (30.2) |
| No | 2600 (37.1) |
| Unknown | 2285 (32.6) |

TABLE 5
Metastatic castrate-resistant prostate cancer cohort

| Characteristic | n (%) |
|---|---|
| All | 118 (100) |
| Age, years | |
| Mean (SD) | 67.2 (8.3) |
| Biopsy site | |
| Lymph node | 50 (42.4) |
| Bone | 31 (26.3) |
| Visceral | 22 (18.6) |
| Other/unknown | 15 (12.7) |
| Prior abiraterone or enzalutamide | |
| No | 61 (51.7) |
| Yes | 54 (45.8) |
| Unknown | 3 (2.5) |
| Prior taxane treatment | |
| Yes | 44 (37.3) |
| No | 71 (60.2) |
| Unknown | 3 (2.5) |

### Model Building

Using previously published data on TCGA, the level of HRD in each tumor was defined based on a mutational signature (HRDetect) previously validated in other tumors types, and TCGA tumors were defined as HRD or HR intact. For gene selection, a list of genes with high expression specificity in prostate cancer was included. A list of 328 genes was provided by Decipher Biosciences found to have low distributional differences based on the Kolmogorov-Smirnov statistic (≤0.25) between both the Mayo Clinic cohort and 32,000 samples from the commercial use of the PCa Decipher test. The genes were ordered by Spearman's correlation with HRDetect and the 100 genes with the highest absolute value correlation in a random two-thirds sample of TCGA samples were selected (Training cohort, n=328). To this, an additional 13 genes were added which were part of the original 328 genes with high reproducibility in PCa, and were also included in a previous study identifying genes that characterize HRD tumors. These 113 genes (Table 6) were used to train a generalized linear model using 5-fold cross validation to adjust alpha=0.5. The final model had 16 genes with non-zero coefficients (Table 7) with higher scores predicting for HRD tumors as HRD_{model}. Receiver-operator curves were produced for the training, validation (remaining 163 samples), and all TCGA samples with expression data. A threshold for model cutoff for defining HRD_{model} using the “cutpointr” command in the “cutpointr” package by maximizing the Youden-Index after kernel smoothing the distributions of the two classes.

TABLE 6
Genes selected for model building

| | | | | |
|---|---|---|---|---|
| ADM | CENPF | DHRS4 | IQCG | XYLB |
| C1orf54 | ASPN | MSR1 | CFDP1 | PDE10A |
| CENPJ | ZNF69 | SKAP2 | CYTH3 | GDF11 |
| CHRNA5 | TOP2A | LINGO3 | TRIM29 | CGREF1 |
| FDPS | KRT14 | KRT5 | OS9 | GATA3 |
| GJB2 | CDKN3 | RSRC1 | EDN3 | RNF122 |
| HIST2H2BE | AZIN1 | LBH | FCGR2A | FST |
| INSIG1 | CDC6 | KRT15 | SCGB1A1 | HSD17B4 |
| MCM7 | TSEN15 | ELP3 | CCK | CHGB |
| NETO2 | EZH2 | CPA6 | GOLGA2 | APOL4 |
| PRPF38A | NDUFAF6 | WDR1 | LY6K | ACSM1 |
| RRM2 | CBX4 | C9orf66 | TNS4 | RAB11FIP3 |
| SFPQ | PROK1 | TTL | PABPC3 | U2AF2 |
| ST6GALNAC2 | CCNB2 | ZFAND1 | RELN | |
| DERL1 | CSPP1 | COL28A1 | NSA2 | |
| TPT1 | HOXC4 | IRX3 | ABHD11 | |
| CENPU | METTL2A | TRIM52 | PTTG1 | |
| GABRD | MCCC1 | NOX4 | PFKFB2 | |
| KCNN4 | CYP3A5 | OTX1 | PRDM12 | |
| ECHDC1 | ITGBL1 | CPXM1 | ARMCX1 | |
| HJURP | SLC16A14 | AMD1 | CA11 | |
| NUSAP1 | TRMT10A | SH3RF2 | ASCL2 | |
| FAM111B | OTUD6B | KIF3A | FOXS1 | |
| SKA3 | DACT2 | STX3 | LY6H | |
| EPHA6 | PPP1R1B | SLC16A9 | HES6 | |

TABLE 7
Coefficients and genes for HRD_{model}

| Gene symbol | Gene name | Coefficient |
|---|---|---|
| FDPS | Farnesyl diphosphate synthase | −0.0806833 |
| GJB2 | Gap junction protein beta 2 | 0.0001539 |
| INSIG1 | Insulin induced gene 1 | −0.0033702 |
| ECHDC1 | Ethylmalonyl-CoA decarboxylase 1 | −0.0418986 |
| TPT1 | Tumor protein, translationally- | −0.1277682 |
| | controlled 1 | |
| DERL1 | Derlin 1 | 0.12971325 |
| NUSAP1 | Nucleolar and spindle | 0.07560427 |
| | associated protein 1 | |
| CCNB2 | Cyclin B2 | 0.1250437 |
| ZNF69 | Zinc finger protein 69 | −0.0263461 |
| TSEN15 | tRNA splicing endonuclease | 0.1562021 |
| | subunit 15 | |
| KCNN4 | Potassium calcium-activated | −0.0306575 |
| | channel subfamily N member 4 | |
| GABRD | Gamma-aminobutyric acid type A | 0.20867555 |
| | receptor delta subunit | |
| HOXC4 | homeobox C4 | 0.06620479 |
| ACTC1 | Actin, alpha, cardiac muscle 1 | 0.03575002 |
| ZNF185 | Zinc finger protein 185 (LIM domain) | 0.04305947 |
| METTL2A | Methyltransferase like 2A | 0.04199085 |

### Expression-Based and Genomic Markers

For the genes used for model building, we performed enrichment analysis of all biological process ontologies using DAVID (https://david.ncifcrf.gov/) and processes with p<0.05 were displayed. We derived expression-based signatures as described by the Hallmark Gene Set Collection to characterize the signature pathways expressed in HRD_{model} tumors for exploratory analysis in the GRID. We also compared fraction genome altered between HRD_{model} and non-HRD_{model} tumors as an orthogonal reflection of genomic instability and potentially HRD. Fraction genome altered was available for TCGA and the mCRPC cohort and was defined as the length of segments with log 2 or linear copy number alteration value larger than 0.2 divided by the length of all segments measured as available in cBioPortal (https://www.cbioportal.org/). The HRD score was defined based previous model incorporating various measure of genomic instability (loss of heterozygosity, telomeric allelic imbalance, and large-scale state transitions) with higher scores suggesting HRD as previously validated and derived in TCGA PCa.

Within TCGA and mCRPC cohorts, we defined tumors with at least one non-silent HR-gene mutation of the 15 genes which would have been qualification for a recent landmark trial assessing PARP inhibition in advanced PCa. Finally, we also calculated the genomic risk score of each tumor in JHMI to include in multivariable metastasis-free survival outcome analysis to optimize regression parsimony. The genomic risk score is a previously validated, expression-based signature (ranging from 0 to 1) with cut-off (>0.6) delineating tumors at higher risk of developing metastatic recurrence following RP.

### Clinical Outcomes

Follow-up was available for TCGA, Mayo Clinic, and JHMI. In TCGA, disease recurrence was defined as either metastatic recurrence or biochemical recurrence based on a measurable serum prostate-specific antigen. Since TCGA was derived from multiple contributing institutions, the follow-up regimens likely vary by patient. Metastatic recurrence in Mayo Clinic and JHMI were based on imaging using computed tomography or bone scan. In Mayo Clinic, patients may have received adjuvant treatments for PCa. Patients from JHMI did not receive any adjuvant treatments and metastatic recurrence was assessed only following biochemical recurrence as defined by a rise in prostate-specific antigen (PSA) >0.2 ng/mL. Given the uniformity of the follow-up management for the patients in JHMI and the value of metastatic recurrence as a strong surrogate endpoint for overall survival, we chose to evaluate HRDmodel tumors in JHMI in multivariable survival analysis.

Within MI-ONCOSEQ, outcomes of response to olaparib were collected, and a PSA decline of 75% of the pre-PARP inhibitor PSA was deemed a strong response. PSA progression was defined as a >25% increase over the nadir PSA while on olaparib.

### Statistical Tests

All statistical analyses were conducted using R version 3.6.2 (Vienna, Austria). All continuous variables were compared by HRD_{model} versus non-HRD_{model} using the two-sided Wilcoxon-Rank Sum test. Chi-squared tests were used to compare factor variables in TCGA. Kaplan-Meier analyses were used to estimate failure-free survival and log-rank tests were used to compare groups. For the JHMI cohort, multivariable Cox-regression including adjustments for tumor characteristics and genomic risk score was used to calculate adjusted hazard ratios for HRD_{model} versus non-HRD_{model} tumors. The Cochrane-Armitage test for trend was used to compare proportion of patients in the mCRPC cohort based on HRD_{model} and groupings of fraction genome altered: low (lowest quartile), average (interquartile), and high (upper quartile). Upper 95% confidence intervals (CIs) for proportions were calculated using the Sison-Glaz method for multinomial proportions using the “MultinomCI” command in the “DescTools” package in R.

### Creating a Model for Homologous Recombination Deficiency

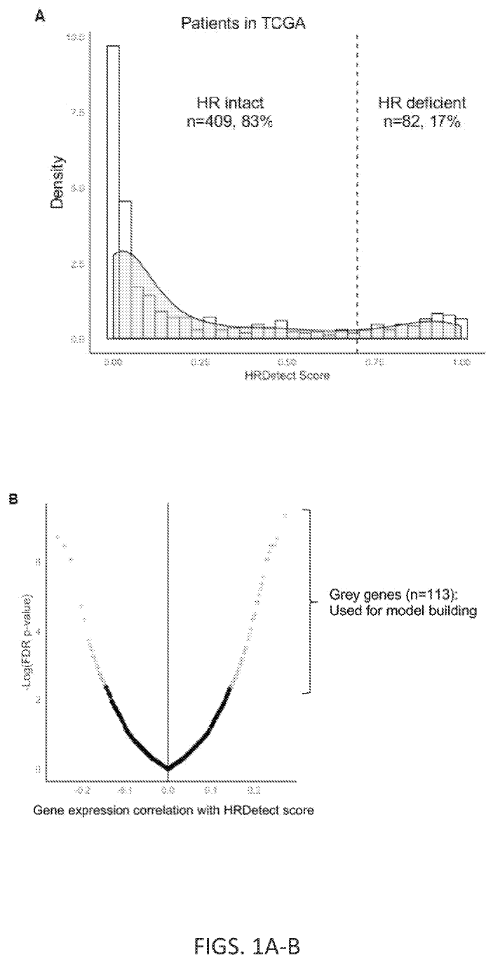

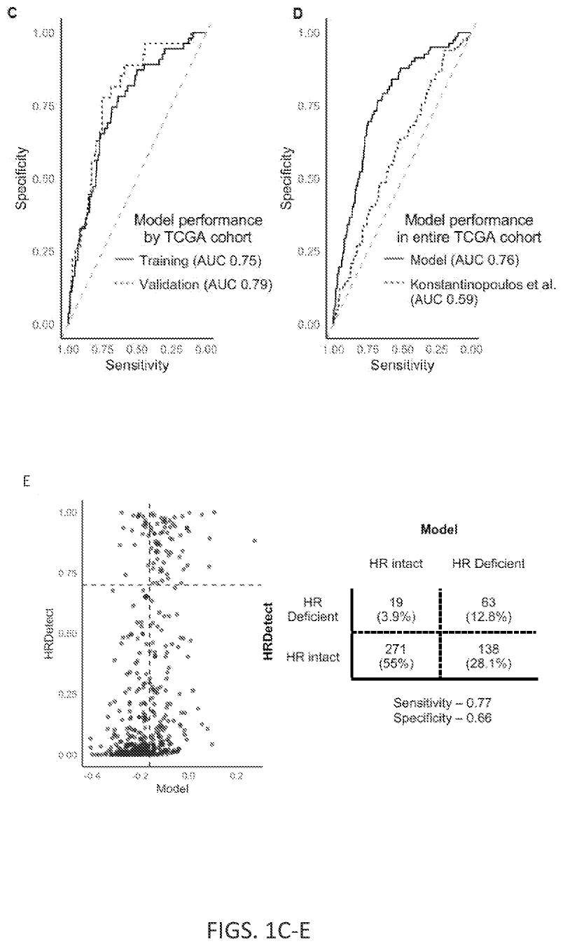

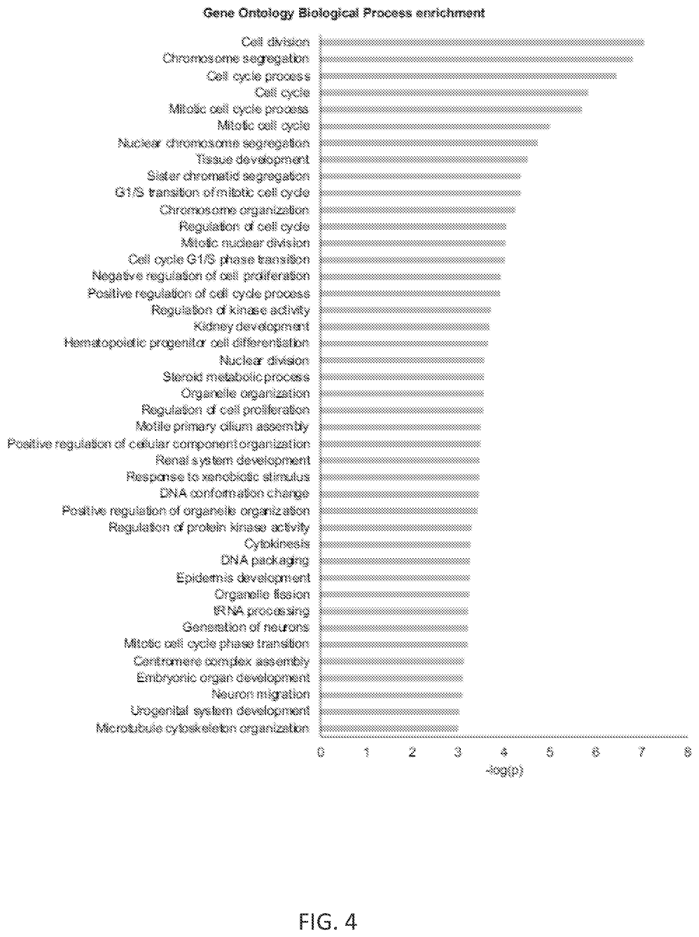

From previous work, we applied the HRDetect mutational signature scores and cutoff (>0.7) to identify 82/491 (17%) HRD tumors in TCGA (FIG. 1A). To prepare for model building, we identified 1198 genes with low distributional difference within PCa (See Methods and Materials). From these, we included the top 100 genes most correlated with HRDetect scores in TCGA. We added an additional 13 genes which also had low distributional difference within PCa and were identified in Peng et al. as part of a list of genes whose expression patterns were used to identify HRD tumors across multiple tumors types (FIG. 1B). Gene ontology analysis of these 113 genes (Table 6) showed enrichment predominantly for processes of cellular proliferation including cell division, chromosome segregation, and cell cycle processes (FIG. 4). These genes were applied to a random two-thirds of TCGA samples to train a model to predict HRD (Table 7). The areas under the curve were similar between training (0.75) and validation (0.79) cohorts (FIG. 1C). Among all TCGA tumors, HRD_{model} was superior to a previous model created for ovarian cancer that predicted HRD tumors (Areas under the curve: 0.76 versus 0.59; FIG. 1D). After optimal cutoff selection to identify HRD_{model} tumors, our prostate transcriptomic model was 77% sensitive and 66% specific for HRD tumors based on DNA sequence level data using HRDetect (FIG. 1E). Together these data suggest a transcriptomic model specific to PCa can classify primary tumors as phenotypically HRD.

### Clinical and Molecular Characteristics of HRD_{model} Tumors

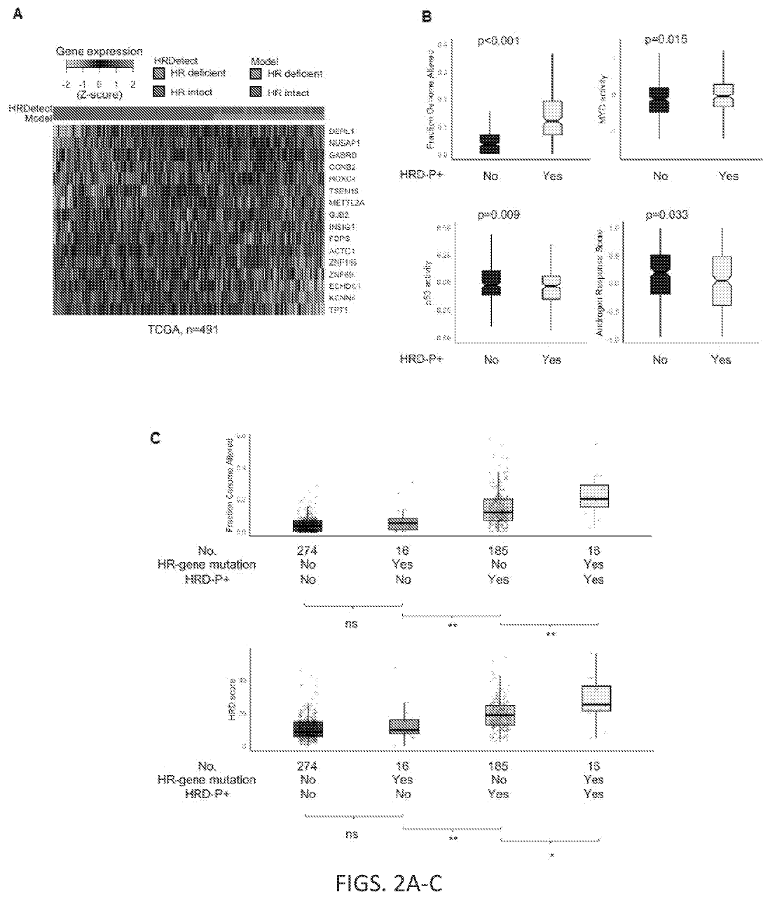

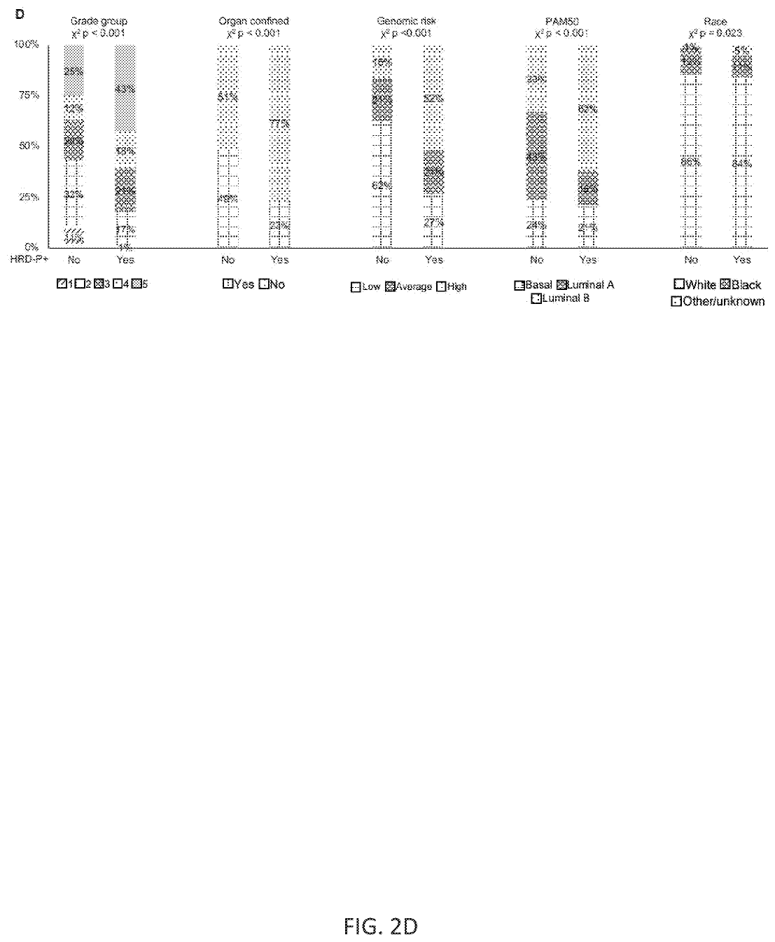

The final model, comprised of 16 genes, identified 201/491 (41%) tumors in TCGA as HRD_{model} (FIG. 2A). Similar to tumors with HRD, HRD_{model} tumors were noted to have higher levels of fraction genome altered—a measure of mutational burden and genomic instability as a potential reflection of HRD—higher levels of MYC activity, and lower levels of p53 activity (FIG. 2B). Specific to PCa, we noted HRD_{model} tumors were predicted to be less responsive to androgen deprivation therapy based on a previously published androgen response signature (FIG. 2B).

Tumors were identified in TCGA with at least one non-silent HR-gene mutation which would have qualified for a recent clinical trial assessing PARP-inhibition (n=32, 6.5%). Among non-HRD_{model} tumors, fraction genome altered and HRD scores (a composite marker of genomic instability with high scores suggesting HRD) did not differ between tumors with and without HR-gene mutations (FIG. 2C). HRD_{model} tumors without HR-gene mutation had higher fraction genome altered and HRD scores than non-HRD_{model} tumors with HR-gene mutation.

Additionally, HRD_{model} tumors with HR-gene mutation had the highest fraction genome altered and HRD scores. We performed linear regression predicting for fraction genome altered and HRD scores with HR-gene mutation, HRD_{model}, and an interaction term between the two regressors. HR-gene mutations were not associated with higher fraction genome altered (Estimate. 0.028, 95% confidence interval [CI]-0.014 to 0.071, p=0.1944), while there was an association between HRD_{model} tumors and high fraction genome altered (Estimate 0.103, 95% CI 0.088 to 0.120, p<0.0001; Table 8). Similarly, HR-gene mutations were not associated with higher HRD scores (Estimate: 2.53, 95% CI −1.88 to 6.93, p=0.2601) while HRD_{model} was (Estimate 8.29, 95% CI 6.66 to 9.92, p<0.0001; Table 9).

TABLE 8
Linear regression for fraction genome altered in TCGA

| Regressor | Estimate (95% CI) | p |
|---|---|---|
| Intercept | 0.045 (0.035 to 0.055) | <0.0001 |
| HRD_{model} | 0.103 (0.088 to 0.120) | <0.0001 |
| (vs not HRD_{model}) | | |
| HR-gene mutation | 0.028 (−0.014 to 0.071) | 0.1944 |
| (vs. no HR-gene mutation) | | |
| Interaction | 0.059 (−0.001 to 0.120) | 0.0558 |
| (HRD_{model}:HR-gene mutation) | | |

TABLE 9
Linear regression for HRD score in TCGA

| Regressor | Estimate (95% CI) | p |
|---|---|---|
| Intercept | 11.28 (10.25 to 12.32) | <0.0001 |
| HRD_{model} | 8.29 (6.66 to 9.92) | <0.0001 |
| (vs not HRD_{model}) | | |
| HR-gene mutation | 2.53 (−1.88 to 6.93) | 0.2601 |
| (vs. no HR-gene mutation) | | |
| Interaction | 6.27 (0.00 to 12.54) | 0.0501 |
| (HRD_{model}:HR-gene mutation) | | |

Within TCGA, HRD_{model} tumors were more aggressive based on tumor grade and stage and a previously validated genomic risk score (FIG. 2D). HRD_{model} tumors were more often classified as Luminal B tumors based on the PAM50 classification which tends to be the subtype for breast cancer tumors with BRCA2 deficiency. HRD_{model} status did not differ substantially by White and Black genomic ancestry as derived from Yuan et al., but HRD_{model} were derived from more patients of other/unknown race (5% vs 1%). From these data, HRD_{model} tumors appear to characteristically resemble tumors with HRD and may predict HRD phenotypes with high fraction genome altered better than single HR-gene mutations.

### Clinical Outcomes and Molecular Pathways for HRD_{model} Tumors in Independent Prostate Cancer Cohorts

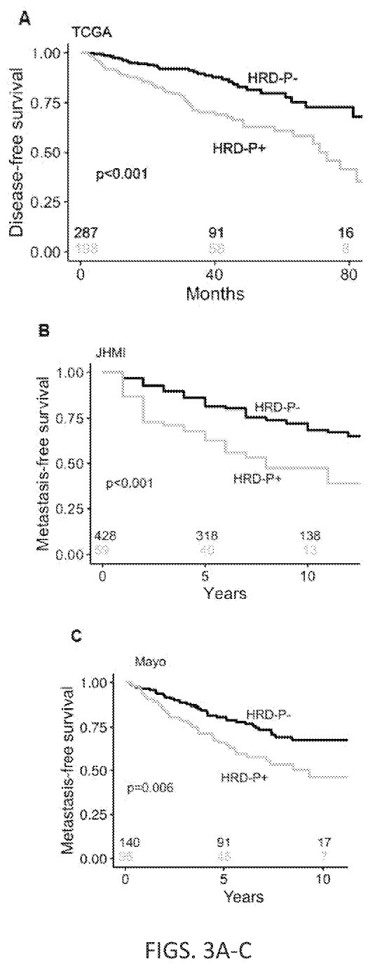

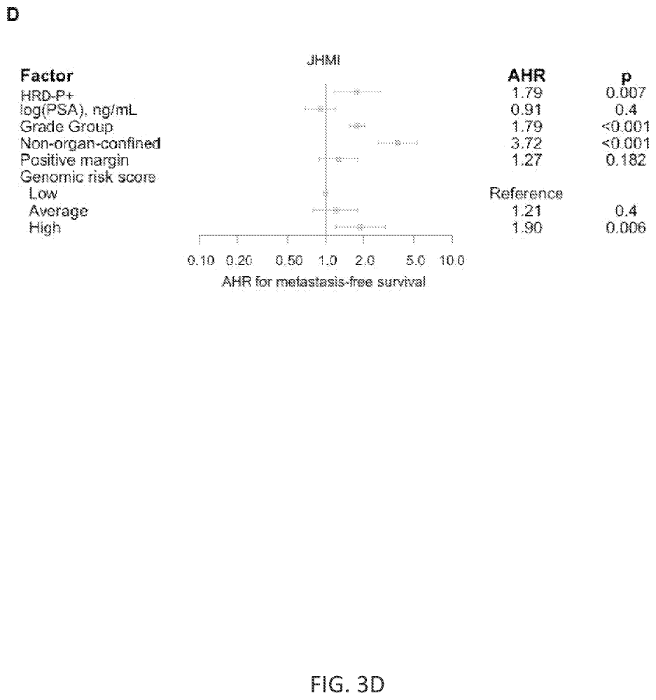

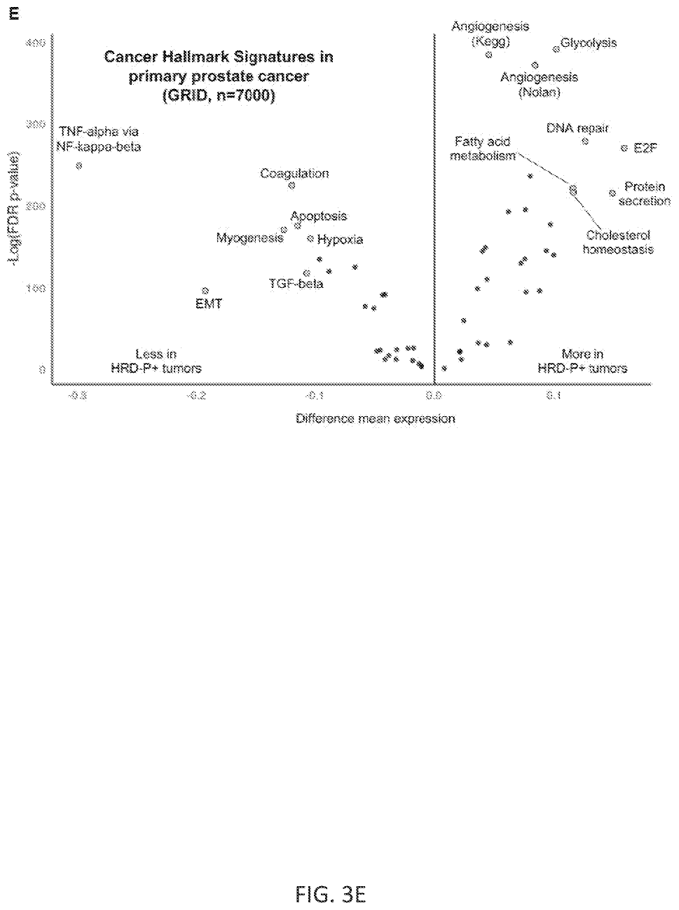

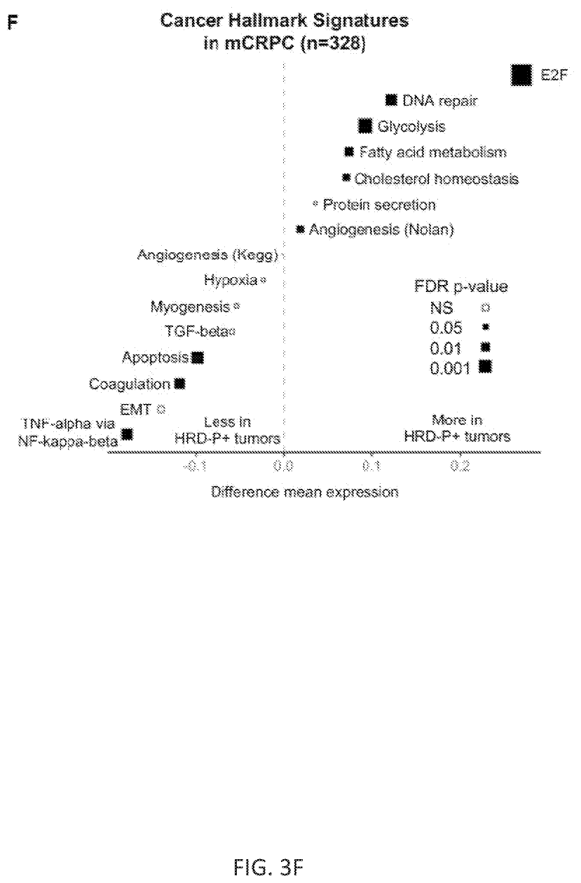

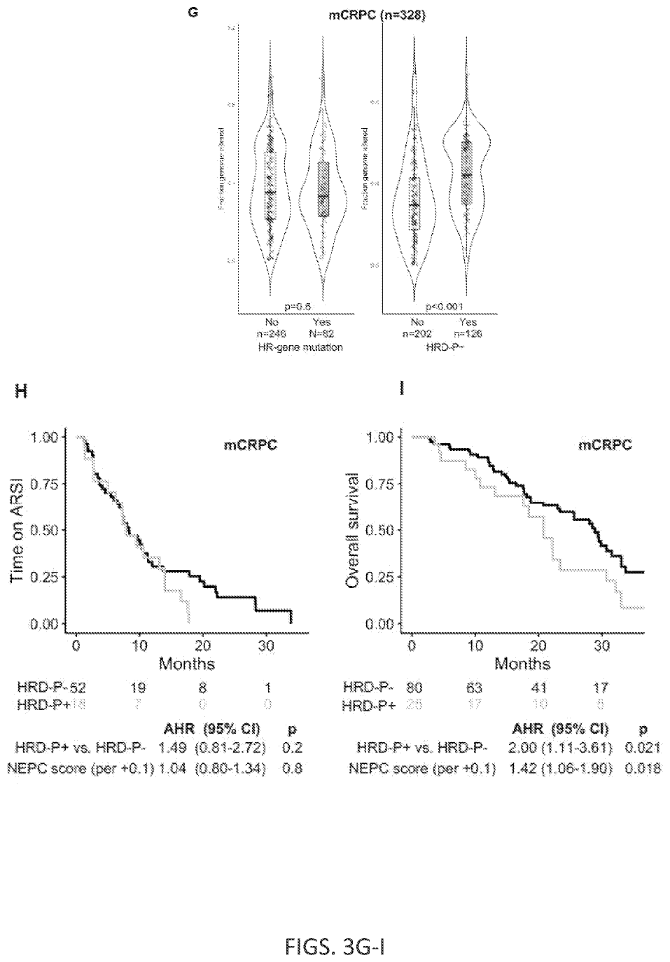

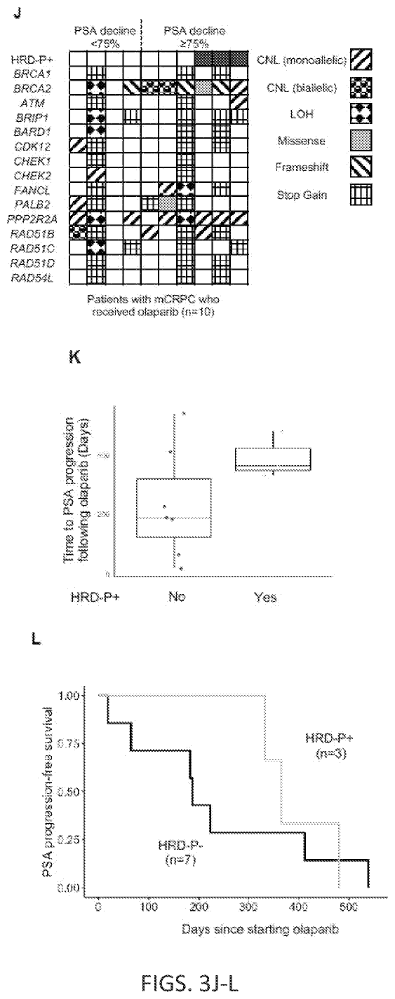

In three independent cohorts of men with primary PCa treated with radical prostatectomy, HRD_{model} tumors were associated with shorter time to cancer recurrence (FIG. 3A-C). In the JHMI cohort which received no adjuvant treatment prior to any metastatic recurrence, HRD_{model} was associated with shorter time to metastatic recurrence even after adjusting for relevant tumor pathology and genomic risk scores (Adjusted hazard ratio: 1.79, 95% CI 1.17-2.72, p=0.007; FIG. 3D). Thus, HRD_{model} tumors have a greater propensity for disease recurrence following surgery.

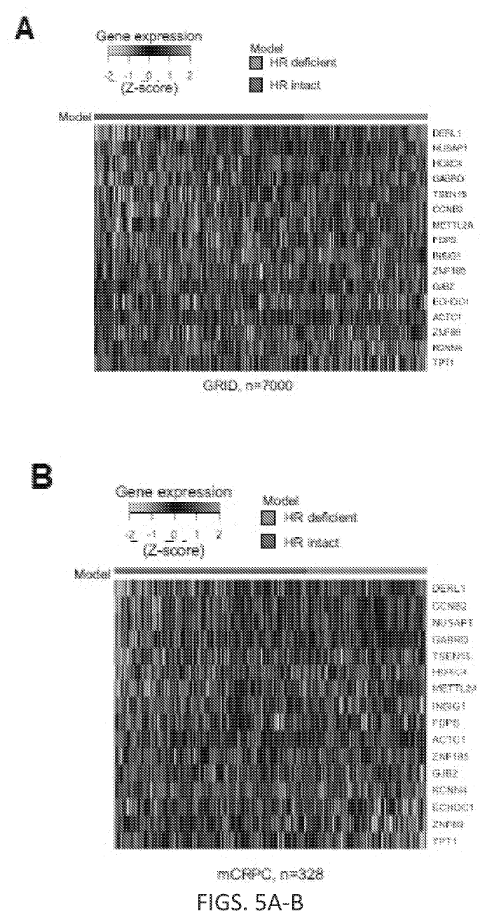

In a prospectively maintained registry of primary PCa (GRID, Table 4 and FIG. 5A), 37%/o (2599/7000) of tumors were HRD_{model}. The expressions of Cancer Hallmark Signatures pathways (16) were compared between HRD_{model} and non-HRD_{model} tumors in this cohort which revealed several differences (FIG. 3E). Notably, markers of angiogenesis, E2F, DNA repair, and various markers of metabolism were increased in HRD_{model} tumors. Meanwhile, apoptosis, among other signatures, was decreased. We then assessed all the signatures from this analysis with −Log(FDR p-value) >250 or mean difference in expression >0.1 or <−0.1 in a cohort of men with mCRPC (Signatures: n=15; FIG. 5B). Of these signatures, angiogenesis and E2F were still found to be increased in HRD_{model} tumors with FDR p<0.05 (FIG. 3F). Finally, we assessed fraction genome altered in mCRPC and found HRD_{model} tumors had higher levels of genomic alterations (FIG. 3G).

Meanwhile, tumors with HR-gene mutations (n=19, 16.1%) were not associated with level of fraction genome altered (FIG. 3H).

Finally, in a cohort of 10 men with mCRPC and RNAseq data who received the PARP inhibitor olaparib, three were determined to be HRD_{model}. This was despite nine of these patients having HR-gene mutations (FIG. 3I). In total, a decrease in serum PSA of at least 75% in response to olaparib was seen in all three men with HRD_{model} tumors (100%) vs three of the seven non-HRD_{model} tumors (43%). Notably, BRCA1 or BRCA2 alterations were present in two of the four men who did not experience a PSA decrease of at least 75% including one with a deep deletion in BRCA2 (FIG. 3I). The three men with HRD_{model} tumors were among those with the longest PSA progression-free survival—each over 300 days (FIG. 3J-K).

These results showed that the methods and genomic classifiers of the disclosure are useful for identifying homologous recombination deficiency in prostate cancer subjects. These results suggested that the methods and markers of the present disclosure would be useful for diagnosing, prognosing, determining the progression of cancer, or predicting benefit from therapy in a subject having prostate cancer. The results further showed that the methods and classifiers are useful for predicting response to therapy. The results also showed that the methods of the disclosure are useful for treating prostate cancer with a PARP inhibitor. The results showed that the methods and classifiers of the disclosure may be used to determine a treatment for a subject with prostate cancer.

What is claimed is:

1. A method comprising: obtaining a biological sample from a subject having prostate cancer, wherein the sample comprises nucleic acids; and detecting the level of expression of a plurality of targets selected from Table 6 or Table 7.

2. A method comprising: a) obtaining or having obtained a nucleic acid expression level of a plurality of targets selected from Table 6 or Table 7, in a biological sample from a subject having prostate cancer; b) prognosing the patient with homologous recombination deficiency prostate cancer based on the nucleic acid expression levels; and c) administering an effective amount of a treatment to the patient based on the prognosis, wherein the treatment is a PARP inhibitor.

3. A method comprising: a) obtaining or having obtained a nucleic acid expression level of a plurality of targets selected from Table 6 or Table 7, in a biological sample from a subject having prostate cancer; b) determining that the patient has homologous recombination deficiency prostate cancer based on the nucleic acid expression levels; and c) administering an effective amount of a treatment to the subject determined to have homologous recombination deficiency prostate cancer; based on the nucleic acid expression levels, wherein the treatment is a PARP inhibitor.

4. The method of any one of the preceding claims, the method further comprises administering an anti-cancer treatment other than a PARP inhibitor to the subject if the expression levels indicate that the subject does not have homologous recombination deficiency prostate cancer.

5. The method of claim 4, wherein the anti-cancer treatment other than a PARP inhibitor is selected from the group consisting of surgery, chemotherapy, radiation therapy, immunotherapy, biological therapy, neoadjuvant chemotherapy, and photodynamic therapy.

6. The method of any one of the preceding claims, wherein the expression level of said target is reduced expression of said target.

7. The method of any one of the preceding claims, wherein the expression level of said target is increased expression of said target.

8. The method of any one of the preceding claims, wherein the level of expression of said target is determined by using a method selected from the group consisting of in situ hybridization, a PCR-based method, an array-based method, an immunohistochemical method, an RNA assay method and an immunoassay method.

9. The method of any one of the preceding claims, wherein the method further comprises determining the level of expression of said plurality of targets using at least one reagent that specifically binds to said targets.

10. The method of any one of the preceding claims, wherein the reagent is selected from the group consisting of a nucleic acid probe, one or more nucleic acid primers, and an antibody.

11. The method of any one of the preceding claims, wherein the target comprises a nucleic acid sequence.

12. The method of any one of the preceding claims, wherein the biological sample is a biopsy.

13. The method of any one of the preceding claims, wherein the biological sample is a urine sample, a blood sample or a prostate tumor sample.

14. The method of any one of the preceding claims, wherein the blood sample is plasma, serum, or whole blood.

15. The method of any one of the preceding claims, wherein the subject is a human.

16. The method of any one of the preceding claims, wherein said measuring the level of expression comprises measuring the level of an RNA transcript.

17. The method of any one of the preceding claims, further comprising administering at least one cancer treatment selected from the group consisting of surgery, radiation therapy, immunotherapy, biological therapy, neoadjuvant chemotherapy, and photodynamic therapy after the androgen deprivation therapy.

18. A kit for identifying, diagnosing and/or prognosing prostate cancer in a subject, the kit comprising agents for detecting the presence or expression levels for a plurality of targets, wherein said plurality of genes comprises one or more genes selected from Table 6 or Table 7.

19. The kit of claim 18, wherein said agents comprise reagents for performing in situ hybridization, a PCR-based method, an array-based method, a sequencing method, an immunohistochemical method, an RNA assay method, or an immunoassay method.

20. The kit of claim 18 or 19, wherein said agents comprise one or more of a microarray, a nucleic acid probe, a nucleic acid primer, or an antibody.

21. The kit of any one of claims 18-20, wherein the kit comprises at least one set of PCR primers capable of amplifying a nucleic acid comprising a sequence of a gene selected from Table 6 or Table 7 or its complement.

22. The kit of any one of claims 18-21, wherein the kit comprises at least one probe capable of hybridizing to a nucleic acid comprising a sequence of a gene selected from Table 6 or Table 7 or its complement.

23. The kit of any one of claims 18-22, further comprising information, in electronic or paper form, comprising instructions on how to determine if a subject is likely to be responsive to anti-cancer therapy.

24. The kit of any one of claims 18-23, further comprising one or more control reference samples.

25. A probe set for diagnosing and/or prognosing prostate cancer in a subject, the probe set comprising a plurality of probes for detecting a plurality of target nucleic acids, wherein the plurality of target nucleic acids comprises one or more gene sequences, or complements thereof, of genes selected from Table 6 or Table 7.

26. The probe set of claim 25, wherein at least one probe is detectably labeled.

27. A kit for detecting, diagnosing and/or prognosing prostate cancer comprising the probe set of claim 25 or 26.

28. A system for analyzing a prostate cancer to provide a diagnosis and/or prognosis to a subject having prostate cancer, the system comprising:
a) the probe set of claim 25 or 26; and
b) a computer model or algorithm for analyzing an expression level or expression profile of the plurality of target nucleic acids hybridized to the plurality of probes in a biological sample from a subject who has prostate cancer and determining that the patient does or does not have homologous recombination deficiency prostate cancer based on the nucleic acid expression levels.

29. A kit for diagnosing and/or prognosing prostate cancer in a subject comprising the system of claim 28.

30. The kit of claim 29, further comprising a computer model or algorithm for designating a treatment modality for the subject.

31. The method, kit, probe set or system of any one of the preceding claims, wherein the plurality of targets comprise or consist of one or more targets selected from Table 6 or Table 7.

32. The method, kit, probe set or system of any one of the preceding claims, wherein the plurality of targets comprise or consist of at least about 2, 3, 4, 5, 6, 7, 8, 9, 10, 11, 12, 13, 14, 15, 16, 17, 18, 19, 20, 25, 30, 35, 40, 45, or 50 targets selected from Table 6 or Table 7.

33. The method, kit, probe set or system of any one of the preceding claims, wherein the plurality of targets comprise or consist of 2-10, 2-16, 8-16, 10-16, 13-16, 2-50, or 25-50 targets.

34. The method, kit, probe set or system of any one of the preceding claims, wherein the plurality of targets comprise or consist of each of the targets from Table 6 and/or Table 7.

35. The method, kit, probe set or system of any one of the preceding claims, wherein the plurality of targets comprise or consist of each of the targets from Table 7.

36. The method, kit, probe set or system of any one of the preceding claims, wherein the plurality of targets comprise or consist of:
GABRD, and TSEN15;
GABRD, TSEN15, and DERL1;
GABRD, TSEN15, DERL1, and TPT1;
GABRD, TSEN15, DERL1, TPT1, and CCNB2;
GABRD, TSEN15, DERL1, TPT1, CCNB2, and FDPS;
GABRD, TSEN15, DERL1, TPT1, CCNB2, FDPS, and NUSAP1;
GABRD, TSEN15, DERL1, TPT1, CCNB2, FDPS, NUSAP1, and HOXC4;
GABRD, TSEN15, DERL1, TPT1, CCNB2, FDPS, NUSAP1, HOXC4, and ZNF185;
GABRD, TSEN15, DERL1, TPT1, CCNB2, FDPS, NUSAP1, HOXC4, ZNF185, and METTL2A;
GABRD, TSEN15, DERL1, TPT1, CCNB2, FDPS, NUSAP1, HOXC4, ZNF185, METTL2A, and ECHDC1;
GABRD, TSEN15, DERL1, TPT1, CCNB2, FDPS, NUSAP1, HOXC4, ZNF185, METTL2A, ECHDC1, and ACTC1;
GABRD, TSEN15, DERL1, TPT1, CCNB2, FDPS, NUSAP1, HOXC4, ZNF185, METTL2A, ECHDC1, ACTC1, and KCNN4;
GABRD, TSEN15, DERL1, TPT1, CCNB2, FDPS, NUSAP1, HOXC4, ZNF185, METTL2A, ECHDC1, ACTC1, KCNN4, and ZNF69;
GABRD, TSEN15, DERL1, TPT1, CCNB2, FDPS, NUSAP1, HOXC4, ZNF185, METTL2A, ECHDC1, ACTC1, KCNN4, ZNF69, and INSIG1; or
GABRD, TSEN15, DERL1, TPT1, CCNB2, FDPS, NUSAP1, HOXC4, ZNF185, METTL2A, ECHDC1, ACTC1, KCNN4, ZNF69, INSIG1, and GJB2.
